SEQUENCE LISTING (182 sequences)

SEQ ID NO: 1 - protein, 10 aa, Homo sapiens
GGNVSSKSAA

SEQ ID NO: 2 - protein, 9 aa, Homo sapiens
TYYRSKWYN

SEQ ID NO: 3 - protein, 17 aa, Homo sapiens
ARSLGEGWFPYFYGMDV

SEQ ID NO: 4 - protein, 8 aa, Homo sapiens
GYTFTSYA

SEQ ID NO: 5 - protein, 8 aa, Homo sapiens
INAGNGNT

SEQ ID NO: 6 - protein, 22 aa, Homo sapiens
ARDFMGYCSSTSCYGVTGAFDI

SEQ ID NO: 7 - protein, 8 aa, Homo sapiens
GYTFTSYY

SEQ ID NO: 8 - protein, 8 aa, Homo sapiens
INPSGGST

SEQ ID NO: 9 - protein, 10 aa, Homo sapiens
AREDWGPFDY

SEQ ID NO: 10 - protein, 8 aa, Homo sapiens
GFTFDDYG

SEQ ID NO: 11 - protein, 8 aa, Homo sapiens
INWNGGST

SEQ ID NO: 12 - protein, 10 aa, Homo sapiens
AREGDGAFDI

SEQ ID NO: 13 - protein, 8 aa, Homo sapiens
GFTFSDYA

SEQ ID NO: 14 - protein, 8 aa, Homo sapiens
ISYDGSQK

SEQ ID NO: 15 - protein, 14 aa, Homo sapiens
AKFPSLNDNVGFDV

SEQ ID NO: 16 - protein, 8 aa, Homo sapiens
GGTFSSYA

SEQ ID NO: 17 - protein, 8 aa, Homo sapiens
IIPIFGTA

SEQ ID NO: 18 - protein, 19 aa, Homo sapiens
ARGNHVLRFLEWLPNWFDP

SEQ ID NO: 19 - protein, 8 aa, Homo sapiens
GYTFTNYG

SEQ ID NO: 20 - protein, 8 aa, Homo sapiens
ISGDAGDT

SEQ ID NO: 21 - protein, 21 aa, Homo sapiens
ARDGEYIPMFRGFDNYYGLDV

SEQ ID NO: 22 - protein, 8 aa, Homo sapiens
GGTFSSYA

SEQ ID NO: 23 - protein, 8 aa, Homo sapiens
IIPIFGTA

SEQ ID NO: 24 - protein, 9 aa, Homo sapiens
ARGVDQVDY

SEQ ID NO: 25 - protein, 9 aa, Homo sapiens
GGSITSSNW

SEQ ID NO: 26 - protein, 7 aa, Homo sapiens
ISHSGST

SEQ ID NO: 27 - protein, 9 aa, Homo sapiens
ARAFHGMDV

SEQ ID NO: 28 - protein, 8 aa, Homo sapiens
GYTFTSYG

SEQ ID NO: 29 - protein, 8 aa, Homo sapiens
ISAYNGNT

SEQ ID NO: 30 - protein, 11 aa, Homo sapiens
ARDGGGGYFDY

SEQ ID NO: 31 - protein, 8 aa, Homo sapiens
GGRFSSNV

SEQ ID NO: 32 - protein, 8 aa, Homo sapiens
IIPIFGTP

SEQ ID NO: 33 - protein, 12 aa, Homo sapiens
ASGSTPFDAFDI

SEQ ID NO: 34 - protein, 8 aa, Homo sapiens
GFTFSDYY

SEQ ID NO: 35 - protein, 8 aa, Homo sapiens
IGTSGSTI

SEQ ID NO: 36 - protein, 12 aa, Homo sapiens
AREATSGTYFDY

SEQ ID NO: 37 - protein, 8 aa, Homo sapiens
GGTFSSYA

SEQ ID NO: 38 - protein, 8 aa, Homo sapiens
IIPIFGTA

SEQ ID NO: 39 - protein, 13 aa, Homo sapiens
ARGPSGSPYGMDV

SEQ ID NO: 40 - protein, 9 aa, Homo sapiens
NRDVGGYDY

SEQ ID NO: 41 - protein, 10 aa, Homo sapiens
SSYTASRTVV

SEQ ID NO: 42 - protein, 8 aa, Homo sapiens
SSSIGSNP

SEQ ID NO: 43 - protein, 12 aa, Homo sapiens
AAWDDSLNGDYV

SEQ ID NO: 44 - protein, 6 aa, Homo sapiens
QSISNF

SEQ ID NO: 45 - protein, 9 aa, Homo sapiens
QQGYSVPLT

SEQ ID NO: 46 - protein, 8 aa, Homo sapiens
GSNIRSNG

SEQ ID NO: 47 - protein, 11 aa, Homo sapiens
AAWDDNLNAWV

SEQ ID NO: 48 - protein, 8 aa, Homo sapiens
TSNIENNA

SEQ ID NO: 49 - protein, 11 aa, Homo sapiens
AVWDDSLNGPV

SEQ ID NO: 50 - protein, 8 aa, Homo sapiens
SSNIGSNT

SEQ ID NO: 51 - protein, 11 aa, Homo sapiens
AAWDDSLNGHV

SEQ ID NO: 52 - protein, 8 aa, Homo sapiens
SSNIGKNA

SEQ ID NO: 53 - protein, 11 aa, Homo sapiens
AAWDDRLKGYV

SEQ ID NO: 54 - protein, 9 aa, Homo sapiens
SSDVGGYNY

SEQ ID NO: 55 - protein, 11 aa, Homo sapiens
SSYTSSSTPYV

SEQ ID NO: 56 - protein, 8 aa, Homo sapiens
SSNIANNA

SEQ ID NO: 57 - protein, 11 aa, Homo sapiens
ATWDDDLNGPI

SEQ ID NO: 58 - protein, 8 aa, Homo sapiens
SSNIGKNI

SEQ ID NO: 59 - protein, 11 aa, Homo sapiens
AAWDDSLRSWL

SEQ ID NO: 60 - protein, 9 aa, Homo sapiens
SSDVGGYNY

SEQ ID NO: 61 - protein, 11 aa, Homo sapiens
SSYTSSSTPWV

SEQ ID NO: 62 - protein, 9 aa, Homo sapiens
SSDVGGYNY

SEQ ID NO: 63 - protein, 11 aa, Homo sapiens
SSYTSSSTPYV

SEQ ID NO: 64 - protein, 6 aa, Homo sapiens
QDISNY

SEQ ID NO: 65 - protein, 9 aa, Homo sapiens
QQYDNLPLT

SEQ ID NO: 66 - protein, 127 aa, Homo sapiens
QLQLQESGPGLVKPSQTLSLTCAISGGNVSSKSAAWNWIRQSPSRGLEWLGRTYYRSKWYNDYAVSVKSRITINPDTSKNQFSLQLKSVTPEDTAVYYCARSLGEGWFPYFYGMDVWGQGTTVTVSS

SEQ ID NO: 67 - protein, 129 aa, Homo sapiens
QVQLVQSGAEVKKPGASVKVSCKASGYTFTSYAMHWVRQAPGQRLEWMGWINAGNGNTKYSQKFQGRVTITRDTSASTAYMELSSLRSEDTAVYYCARDFMGYCSSTSCYGVTGAFDIWGQGTMVTVSS

SEQ ID NO: 68 - protein, 117 aa, Homo sapiens
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCAREDWGPFDYWGQGTTVTVSS

SEQ ID NO: 69 - protein, 117 aa, Homo sapiens
QVQLVESGGGVVRPGGSLRLSCAASGFTFDDYGMSWVRQAPGKGLEWVSGINWNGGSTGYADSVKGRFTISRDNAKNSLYLQMNSLRAEDTALYYCAREGDGAFDIWGQGTLVTVSS

SEQ ID NO: 70 - protein, 121 aa, Homo sapiens
QVQLVETGGGVVQPGRSLRLSCAASGFTFSDYAMHWVRQAPGKGLEWVTLISYDGSQKYYADSVKGRFTISRDDSKNTVDLQMNSLKAEDTAVYYCAKFPSLNDNVGFDVWGQGTMVTVSS

SEQ ID NO: 71 - protein, 118 aa, Homo sapiens
QVQLMQSGAEVKKPGSSVKVSCKASGGTFSSYAISWVRQAPGQGLEWMGGIIPIFGTANYAQKFQGRVTITADESTSTAYMELSSLRSEDTAVYYCARGNHVLRFLEWLPNWFDPWGQ

SEQ ID NO: 72 - protein, 128 aa, Homo sapiens
QVQLVQSGAEVKKPGASVKVSCKASGYTFTNYGISWVRQAPGQGLEWMGWISGDAGDTKFAQKFQGRVTMTTDTSTTTTYMELRSLRSDDTAVYYCARDGEYIPMFRGFDNYYGLDVWGQGTTVTVSS

SEQ ID NO: 73 - protein, 116 aa, Homo sapiens
EVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGVDQVDYWGQGTLVTVSS

SEQ ID NO: 74 - protein, 116 aa, Homo sapiens
QVQLQESGPGLVKPSGTLSLTCAVSGGSITSSNWWSWVRQPPGKGLEWIGDISHSGSTNYNPSLKGRVTMSVDRSKNQFFLKLSSVTAADTAMYYCARAFHGMDVWGQGTTVTVSS

SEQ ID NO: 75-78 - skipped (4 entries)

SEQ ID NO: 79 - protein, 110 aa, Homo sapiens
QSVLTQPASVSGSPGQSITISCTGTNRDVGGYDYVSWYQQHPGKAPKLMIYGVSNRPSGISNRFSGSKSGSTASLTISGLQVEDEAAYYCSSYTASRTVVFGGGTKVTVL

SEQ ID NO: 80 - protein, 111 aa, Homo sapiens
QSVLTQPPSASGTPGQRVTISCSGSSSSIGSNPVNWYQQLPGTAPKLLIYSNNQRPSGVPDRFSGSKSGTSPSLAISGLQSEDEADYYCAAWDDSLNGDYVFGTGTKVTVL

SEQ ID NO: 81 - protein, 107 aa, Homo sapiens
DIQMTQSPSSLSASVGDRVTITCRASQSISNFVNWYQQKPGEVPKLLIYGASSLQSGVPSRFSGSGSGTDFTLTISSLQPEDFATYYCQQGYSVPLTFGGGTKVEIK

SEQ ID NO: 82 - protein, 110 aa, Homo sapiens
QAVVTQPPSASGTPGQRVTISCSGSGSNIRSNGVTWYQQFPGTAPKLLIYDNNQRPSGVPGRFSGSKSGTSGSLAISGLQSEDEADYYCAAWDDNLNAWVFGAGTQLTVL

SEQ ID NO: 83 - protein, 110 aa, Homo sapiens
QSVLTQPPSVSGAPRERVTISCSGSTSNIENNAVNWYQQLPGKAPKLLIYYDDLVPSGVSDRFSGSKSGTSASLAISGLQSGDEAYYYCAVWDDSLNGPVFGGGTKVTVL

SEQ ID NO: 84 - protein, 110 aa, Homo sapiens
QSVLTQPPSASGTPGQRVTISCSGSSSNIGSNTVNWYQQFPGTAPKLLIYSNNQRPSGVPDRFSGSKSGTSASLAISGLQSEDEADYYCAAWDDSLNGHVFGTGTKVTVL

SEQ ID NO: 85 - protein, 110 aa, Homo sapiens
QPVLTQPPSVSEAPRQRVTISCSGSSSNIGKNAVNWYQQFPGKAPKLLIYYDDLLPSGVSDRFSGSRSGTSASLAISGLQSEDEADYYCAAWDDRLKGYVFGTGTQLTVL

SEQ ID NO: 86 - protein, 111 aa, Homo sapiens
QAVVTQPASVSGSPGQSITISCTGTSSDVGGYNYVSWYQQHPGKAPKLMIYDVSNRPSGVSNRFSGSKSGNTASLTISGLQAEDEADYYCSSYTSSSTPYVFGTGTKVTVL

SEQ ID NO: 87 - protein, 110 aa, Homo sapiens
QSVLTQPPSVSEAPRQRVTISCSGSSSNIANNAVNWYQQLPGKAPKLLIYYDDLLASGVSDRFSGSRSGTSAALAISGLQSEDEADYYCATWDDDLNGPIFGGGTKVTVL

SEQ ID NO: 88 - protein, 110 aa, Homo sapiens
QAVVTQPPSASGTPGQRITISCSGSSSNIGKNIVNWYQQVPGAAPKLLIYNNSQRPSGVPDRFSGSKSGTSASLAISGLQSEDEADYYCAAWDDSLRSWLFGGGTKVTVL

SEQ ID NO: 89 - protein, 111 aa, Homo sapiens
QPVLTQPASVSGSPGQSITISCTGTSSDVGGYNYVSWYQQHPGKAPKLMIYEVSNRPSGVSNRFSGSKSGNTASLTISGLQAEDEADYYCSSYTSSSTPWVFGGGTKVTVL

SEQ ID NO: 90 - protein, 111 aa, Homo sapiens
QAVVTQPASVSGSPGQSITISCTGTSSDVGGYNYVSWYQQHPGKAPKLMIYEVTNRPSGVSNRFSGSKSGNTASLTISGLQAEDEADYYCSSYTSSSTPYVFGTGTKVTVL

SEQ ID NO: 91 - protein, 107 aa, Homo sapiens
DIQMTQSPSSLSASVGDRVTITCQASQDISNYLNWYQQKPGKAPKLLIYDASNLETGVPSRFSGSGSGTDFTFTISSLQPEDIATYYCQQYDNLPLTFGGGTKVDIK

SEQ ID NO: 92 - DNA, 30 bp, Homo sapiens
  source 1..30: mol_type = other DNA
gggggcaatgtctctagtaaaagtgctgct

SEQ ID NO: 93 - DNA, 27 bp, Homo sapiens
  source 1..27: mol_type = other DNA
acatactacaggtccaagtggtataac

SEQ ID NO: 94 - DNA, 51 bp, Homo sapiens
  source 1..51: mol_type = other DNA
gcaagaagcctgggtgagggctggttcccatacttctacggtatggacgtc

SEQ ID NO: 95 - DNA, 24 bp, Homo sapiens
  source 1..24: mol_type = other DNA
ggatacaccttcactagctatgct

SEQ ID NO: 96 - DNA, 24 bp, Homo sapiens
  source 1..24: mol_type = other DNA
atcaacgctggcaatggtaacaca

SEQ ID NO: 97 - DNA, 66 bp, Homo sapiens
  source 1..66: mol_type = other DNA
gcgagagattttatgggatattgtagtagtaccagctgctatggggttactggtgcttttgatatc

SEQ ID NO: 98 - DNA, 24 bp, Homo sapiens
  source 1..24: mol_type = other DNA
ggatacaccttcaccagctactat

SEQ ID NO: 99 - DNA, 24 bp, Homo sapiens
  source 1..24: mol_type = other DNA
atcaaccctagtggtggtagcaca

SEQ ID NO: 100 - DNA, 30 bp, Homo sapiens
  source 1..30: mol_type = other DNA
gcgagagaggattggggtccctttgactac

SEQ ID NO: 101 - DNA, 24 bp, Homo sapiens
  source 1..24: mol_type = other DNA
ggattcacctttgatgattatggc

SEQ ID NO: 102 - DNA, 24 bp, Homo sapiens
  source 1..24: mol_type = other DNA
attaattggaatggtggtagcaca

SEQ ID NO: 103 - DNA, 30 bp, Homo sapiens
  source 1..30: mol_type = other DNA
gcgagagagggggacggtgcttttgatatc

SEQ ID NO: 104 - DNA, 24 bp, Homo sapiens
  source 1..24: mol_type = other DNA
ggattcaccttcagtgactatgct

SEQ ID NO: 105 - DNA, 24 bp, Homo sapiens
  source 1..24: mol_type = other DNA
atatcatatgatggaagtcaaaaa

SEQ ID NO: 106 - DNA, 42 bp, Homo sapiens
  source 1..42: mol_type = other DNA
gcgaagtttccgtccttaaatgacaacgtgggttttgatgtc

SEQ ID NO: 107 - DNA, 24 bp, Homo sapiens
  source 1..24: mol_type = other DNA
ggaggcaccttcagcagctatgct

SEQ ID NO: 108 - DNA, 24 bp, Homo sapiens
  source 1..24: mol_type = other DNA
atcatccctatctttggtacagca

SEQ ID NO: 109 - DNA, 57 bp, Homo sapiens
  source 1..57: mol_type = other DNA
gcgagaggtaaccacgtattacgatttttggagtggttacccaactggttcgacccc

SEQ ID NO: 110 - DNA, 24 bp, Homo sapiens
  source 1..24: mol_type = other DNA
ggttacacctttaccaactatggt

SEQ ID NO: 111 - DNA, 24 bp, Homo sapiens
  source 1..24: mol_type = other DNA
atcagcggtgacgctggtgacaca

SEQ ID NO: 112 - DNA, 63 bp, Homo sapiens
  source 1..63: mol_type = other DNA
gcgagagatggggaatatattcctatgtttcggggatttgacaactactacggtctggacgtc

SEQ ID NO: 113 - DNA, 24 bp, Homo sapiens
  source 1..24: mol_type = other DNA
ggaggcaccttcagcagctatgct

SEQ ID NO: 114 - DNA, 24 bp, Homo sapiens
  source 1..24: mol_type = other DNA
atcatccctatctttggtacagca

SEQ ID NO: 115 - DNA, 27 bp, Homo sapiens
  source 1..27: mol_type = other DNA
gcgagaggcgtggaccaggttgactac

SEQ ID NO: 116 - DNA, 27 bp, Homo sapiens
  source 1..27: mol_type = other DNA
ggtggctccatcaccagtagtaactgg

SEQ ID NO: 117 - DNA, 21 bp, Homo sapiens
  source 1..21: mol_type = other DNA
atctctcatagtgggagcacc

SEQ ID NO: 118 - DNA, 27 bp, Homo sapiens
  source 1..27: mol_type = other DNA
gcgagagcctttcacggtatggacgtc

SEQ ID NO: 119 - DNA, 24 bp, Homo sapiens
  source 1..24: mol_type = other DNA
ggttacacctttaccagctatggt

SEQ ID NO: 120 - DNA, 24 bp, Homo sapiens
  source 1..24: mol_type = other DNA
atcagcgcttacaatggtaacaca

SEQ ID NO: 121 - DNA, 33 bp, Homo sapiens
  source 1..33: mol_type = other DNA
gcgagagatgggggtggcgggtactttgactac

SEQ ID NO: 122 - DNA, 24 bp, Homo sapiens
  source 1..24: mol_type = other DNA
ggaggcaggttcagcagcaatgtt

SEQ ID NO: 123 - DNA, 24 bp, Homo sapiens
  source 1..24: mol_type = other DNA
atcatccctatttttggtacacca

SEQ ID NO: 124 - DNA, 36 bp, Homo sapiens
  source 1..36: mol_type = other DNA
gcgagcggtagtacgccttttgatgcttttgatatc

SEQ ID NO: 125 - DNA, 24 bp, Homo sapiens
  source 1..24: mol_type = other DNA
ggattcaccttcagtgactactat

SEQ ID NO: 126 - DNA, 24 bp, Homo sapiens
  source 1..24: mol_type = other DNA
attggtactagtggtagtaccata

SEQ ID NO: 127 - DNA, 36 bp, Homo sapiens
  source 1..36: mol_type = other DNA
gcgagagaggccacctctggaacctactttgactac

SEQ ID NO: 128 - DNA, 24 bp, Homo sapiens
  source 1..24: mol_type = other DNA
ggaggcaccttcagcagctatgct

SEQ ID NO: 129 - DNA, 24 bp, Homo sapiens
  source 1..24: mol_type = other DNA
atcatccctatctttggtacagca

SEQ ID NO: 130 - DNA, 39 bp, Homo sapiens
  source 1..39: mol_type = other DNA
gcgagaggtccgagtggtagcccttacggtatggacgtc

SEQ ID NO: 131 - DNA, 27 bp, Homo sapiens
  source 1..27: mol_type = other DNA
aacagagacgttggtggttatgactat

SEQ ID NO: 132 - DNA, 30 bp, Homo sapiens
  source 1..30: mol_type = other DNA
tcttcatacacagccagccgcactgtggtg

SEQ ID NO: 133 - DNA, 24 bp, Homo sapiens
  source 1..24: mol_type = other DNA
agctccagcatcggaagtaatcct

SEQ ID NO: 134 - DNA, 36 bp, Homo sapiens
  source 1..36: mol_type = other DNA
gcagcatgggatgacagcctgaatggcgattatgtc

SEQ ID NO: 135 - DNA, 18 bp, Homo sapiens
  source 1..18: mol_type = other DNA
cagagcataagcaacttt

SEQ ID NO: 136 - DNA, 27 bp, Homo sapiens
  source 1..27: mol_type = other DNA
caacagggttacagtgtcccactcact

SEQ ID NO: 137 - DNA, 24 bp, Homo sapiens
  source 1..24: mol_type = other DNA
ggctccaacatcagaagtaatggt

SEQ ID NO: 138 - DNA, 33 bp, Homo sapiens
  source 1..33: mol_type = other DNA
gcagcatgggatgacaacctgaatgcttgggtg

SEQ ID NO: 139 - DNA, 24 bp, Homo sapiens
  source 1..24: mol_type = other DNA
acctccaatatcgaaaataatgct

SEQ ID NO: 140 - DNA, 33 bp, Homo sapiens
  source 1..33: mol_type = other DNA
gcagtttgggatgacagcctgaatggtccggtt

SEQ ID NO: 141 - DNA, 24 bp, Homo sapiens
  source 1..24: mol_type = other DNA
agctccaacatcggaagtaatact

SEQ ID NO: 142 - DNA, 33 bp, Homo sapiens
  source 1..33: mol_type = other DNA
gcagcatgggatgacagcctgaatggtcatgtc

SEQ ID NO: 143 - DNA, 24 bp, Homo sapiens
  source 1..24: mol_type = other DNA
agctccaacatcggaaagaatgct

SEQ ID NO: 144 - DNA, 33 bp, Homo sapiens
  source 1..33: mol_type = other DNA
gcagcatgggatgacagactgaaaggatacgtc

SEQ ID NO: 145 - DNA, 27 bp, Homo sapiens
  source 1..27: mol_type = other DNA
agcagtgacgttggtggttataactat

SEQ ID NO: 146 - DNA, 33 bp, Homo sapiens
  source 1..33: mol_type = other DNA
agctcatatacaagcagcagcaccccttatgtc

SEQ ID NO: 147 - DNA, 24 bp, Homo sapiens
  source 1..24: mol_type = other DNA
agctccaatatcgcaaataatgct

SEQ ID NO: 148 - DNA, 33 bp, Homo sapiens
  source 1..33: mol_type = other DNA
gcaacatgggatgacgacctgaatggtccgata

SEQ ID NO: 149 - DNA, 24 bp, Homo sapiens
  source 1..24: mol_type = other DNA
agctccaacatcggaaaaaatatt

SEQ ID NO: 150 - DNA, 33 bp, Homo sapiens
  source 1..33: mol_type = other DNA
gcagcatgggatgacagcctgagatcttggctg

SEQ ID NO: 151 - DNA, 27 bp, Homo sapiens
  source 1..27: mol_type = other DNA
agcagtgacgttggtggttataactat

SEQ ID NO: 152 - DNA, 33 bp, Homo sapiens
  source 1..33: mol_type = other DNA
agctcatatacaagcagcagcactccttgggtg

SEQ ID NO: 153 - DNA, 27 bp, Homo sapiens
  source 1..27: mol_type = other DNA
agcagtgacgttggtggttataactat

SEQ ID NO: 154 - DNA, 33 bp, Homo sapiens
  source 1..33: mol_type = other DNA
agctcatatacaagcagcagcaccccgtatgtc

SEQ ID NO: 155 - DNA, 18 bp, Homo sapiens
  source 1..18: mol_type = other DNA
caggacattagcaactat

SEQ ID NO: 156 - DNA, 27 bp, Homo sapiens
  source 1..27: mol_type = other DNA
caacagtatgataatctccccctcact

SEQ ID NO: 157 - DNA, 381 bp, Homo sapiens
  source 1..381: mol_type = other DNA
cagctgcagctgcaggagtcaggtccaggactggtgaagccctcgcagaccctctcactcacctgtgccatctccgggggcaatgtctctagtaaaagtgctgcttggaactggatcaggcagtccccatcgcgaggccttgagtggctgggaaggacatactacaggtccaagtggtataacgattatgcagtctctgtgaaaagtcgaataaccatcaacccagacacatccaagaaccagttctccctgcagttgaagtctgtgactcccgaggacacggctgtgtattactgtgcaagaagcctgggtgagggctggttcccatacttctacggtatggacgtctggggccaagggaccacggtcaccgtctcctca

SEQ ID NO: 158 - DNA, 387 bp, Homo sapiens
  source 1..387: mol_type = other DNA
caggtccagcttgtgcagtctggggctgaggtgaagaagcctggggcctcagtgaaggtttcctgcaaggcttctggatacaccttcactagctatgctatgcattgggtgcgccaggcccccggacaaaggcttgagtggatgggatggatcaacgctggcaatggtaacacaaaatattcacagaagttccagggcagagtcaccattaccagggacacatccgcgagcacagcctacatggagctgagcagcctgagatctgaagacacggctgtgtattactgtgcgagagattttatgggatattgtagtagtaccagctgctatggggttactggtgcttttgatatctggggccaagggacaatggtcactgtctcctca

SEQ ID NO: 159 - DNA, 351 bp, Homo sapiens
  source 1..351: mol_type = other DNA
caggtgcagctggtgcagtctggggctgaggtgaagaagcctggggcctcagtgaaggtttcctgcaaggcatctggatacaccttcaccagctactatatgcactgggtgcgacaggcccctggacaagggcttgagtggatgggaataatcaaccctagtggtggtagcacaagctacgcacagaagttccagggcagagtcaccatgaccagggacacgtccacgagcacagtctacatggagctgagcagcctgagatctgaggacacggccgtgtattactgtgcgagagaggattggggtccctttgactactggggccaggggaccacggtcaccgtctcttca

SEQ ID NO: 160 - DNA, 351 bp, Homo sapiens
  source 1..351: mol_type = other DNA
caggtgcagctggtggagtctgggggaggtgtggtacggcctggggggtccctgagactctcctgtgcagcctctggattcacctttgatgattatggcatgagctgggtccgccaagctccagggaaggggctggagtgggtctctggtattaattggaatggtggtagcacaggttatgcagactctgtgaagggccgattcaccatctccagagacaacgccaagaactccctgtatctgcaaatgaacagtctgagagccgaggacacggccttgtattactgtgcgagagagggggacggtgcttttgatatctggggccaaggaaccctggtcaccgtctcctca

SEQ ID NO: 161 - DNA, 363 bp, Homo sapiens
  source 1..363: mol_type = other DNA
caggtgcagctggtggagactgggggaggcgtggtccagcctggaaggtccctgagactctcgtgtgcagcctctggattcaccttcagtgactatgctatgcactgggtccgccaggctccaggcaaggggctggagtgggtgacacttatatcatatgatggaagtcaaaaatattacgcagactccgtgaagggccgattcaccatctccagagacgattccaagaacacggtggatctgcaaatgaacagcctgaaagctgaggacacggctgtgtattactgtgcgaagtttccgtccttaaatgacaacgtgggttttgatgtctggggccaggggacaatggtcaccgtctcctca

SEQ ID NO: 162 - DNA, 378 bp, Homo sapiens
  source 1..378: mol_type = other DNA
caggtgcagttgatgcagtctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttggtacagcaaactacgcacagaagttccagggcagagtcacgattaccgcggacgaatccacgagcacagcctacatggagctgagcagcctgagatctgaggacacggccgtgtattactgtgcgagaggtaaccacgtattacgatttttggagtggttacccaactggttcgacccctggggccaaggaaccctggtcaccgtctcctca

SEQ ID NO: 163 - DNA, 384 bp, Homo sapiens
  source 1..384: mol_type = other DNA
caggttcagcttgtgcagtctggagctgaggtgaagaagcctggggcctcagtgaaggtctcctgcaaggcttctggttacacctttaccaactatggtatcagttgggtgcgacaagccccgggacaagggcttgagtggatgggctggatcagcggtgacgctggtgacacaaaatttgcacagaagttccagggcagagtcaccatgacgacagacacatccacgactacaacgtacatggagctgaggagcctgagatctgacgacacggccgtgtattactgtgcgagagatggggaatatattcctatgtttcggggatttgacaactactacggtctggacgtctggggccaagggaccacggtcactgtctcctca

SEQ ID NO: 164 - DNA, 348 bp, Homo sapiens
  source 1..348: mol_type = other DNA
gaagtgcagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttggtacagcaaactacgcacagaagttccagggcagagtcacgattaccgcggacgaatccacgagcacagcctacatggagctgagcagcctgagatctgaggacacggccgtgtattactgtgcgagaggcgtggaccaggttgactactggggccagggaaccctggtcaccgtctcctca

SEQ ID NO: 165 - DNA, 348 bp, Homo sapiens
  source 1..348: mol_type = other DNA
caggtgcagctacaggagtcgggcccaggactggtgaagccttcggggaccctatccctcacctgcgctgtctctggtggctccatcaccagtagtaactggtggagttgggtccgccagcccccagggaaggggctggagtggattggcgatatctctcatagtgggagcaccaactacaacccgtccctcaagggtcgagtcaccatgtcagtggacaggtccaagaaccagttcttcctgaagctgagttctgtgaccgccgcggacacggccatgtattactgtgcgagagcctttcacggtatggacgtctggggccaagggaccacggtcaccgtctcttca

SEQ ID NO: 166 - DNA, 354 bp, Homo sapiens
  source 1..354: mol_type = other DNA
caggttcagcttgtgcagtctggagctgaggtgaagaagcctggggcctcagtgaaggtctcctgcaaggcttctggttacacctttaccagctatggtatcagctgggtgcgacaggcccctggacaagggcttgagtggatgggatggatcagcgcttacaatggtaacacaaactatgcacagaagctccagggcagagtcaccatgaccacagacacatccacgagcacagcctacatggagctgaggagcctgagatctgacgacacggccgtgtattactgtgcgagagatgggggtggcgggtactttgactactggggccagggaaccctggtcaccgtctcctca

SEQ ID NO: 167 - DNA, 357 bp, Homo sapiens
  source 1..357: mol_type = other DNA
caggtgcagttgatgcagtctggggctgaggtgaagaagcctgggtcctcggtgaaggtctcctgcaaggcttctggaggcaggttcagcagcaatgttatgagctgggtgcgacaggcccctggacaagggcttgagtggatgggagggatcatccctatttttggtacaccaaagtacgcacagaagttccagggcagagtcacgattaccgcggacaaatccacgagcacagcctacatggagctgagcagcctgagatctgaggacacggccgtgtattactgtgcgagcggtagtacgccttttgatgcttttgatatctggggccaagggacaatggtcaccgtctcttca

SEQ ID NO: 168 - DNA, 357 bp, Homo sapiens
  source 1..357: mol_type = other DNA
caggtgcagctggtggagtctgggggaggcttggtcaagcctggagggtccctgagactctcctgtggaatttctggattcaccttcagtgactactatatgagctggatccgccaggctccagggaaggggctggagtgggtttcattcattggtactagtggtagtaccatatactacgcagactctgtgaagggccgattcaccatctccagggacaacgccaataactcactgtttcttcaaatgaacagcctgacagtcgaagacacggccgtgtattactgtgcgagagaggccacctctggaacctactttgactactggggccagggaaccctggtcagcgtctcctca

SEQ ID NO: 169 - DNA, 360 bp, Homo sapiens
  source 1..360: mol_type = other DNA
caggtgcagctacagcagtctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttggtacagcaaactacacacagaagttccagggcagagtcacgattaccgcggacgaatccacgagcacagcctacatggagctgagcagcctgagatctgaggacacggccgtgtattactgtgcgagaggtccgagtggtagcccttacggtatggacgtctggggccaagggaccacggtcatcgtctcctca

SEQ ID NO: 170 - DNA, 330 bp, Homo sapiens
  source 1..330: mol_type = other DNA
cagtctgtgctgactcagcctgcctccgtgtcagggtctcctggacagtcgatcaccatctcctgcactgggaccaacagagacgttggtggttatgactatgtctcctggtaccaacaacacccaggcaaagcccccaaactcatgatttatggtgtcagtaatcggccctcagggatttctaatcgcttctctggctccaagtctggcagcacggcctccctgaccatctctggccttcaggttgaggacgaggctgcttattactgctcttcatacacagccagccgcactgtggtgttcggcggagggaccaaggtcaccgtccta

SEQ ID NO: 171 - DNA, 333 bp, Homo sapiens
  source 1..333: mol_type = other DNA
cagtctgtgctgactcagccaccctcagcgtctgggacccccgggcagagggtcaccatctcttgttctggaagcagctccagcatcggaagtaatcctgtaaactggtaccagcagctcccaggaacggcccccaaactcctcatctatagtaataatcagcggccctcaggggtccctgaccgattctctggctccaagtctggcacctcaccctccctggccatcagtgggctccagtctgaggatgaggctgattattactgtgcagcatgggatgacagcctgaatggcgattatgtcttcggaactgggaccaaggtcaccgtccta

SEQ ID NO: 172 - DNA, 321 bp, Homo sapiens
  source 1..321: mol_type = other DNA
gacatccagatgacccagtctccatcctccctgtctgcatctgtaggagacagagtcaccatcacttgccgggcaagtcagagcataagcaactttgtaaattggtatcagcagaaaccaggggaagtccctaagctcctgatctacggtgcatccagtttgcagagtggggtcccatcaaggttcagtggcagtggatctgggacggatttcactctcaccatcagcagtctgcaacctgaagattttgcgacttactactgtcaacagggttacagtgtcccactcactttcggcggagggaccaaggtggaaatcaaa

SEQ ID NO: 173 - DNA, 330 bp, Homo sapiens
  source 1..330: mol_type = other DNA
caggctgtggtgactcagccaccctcagcgtctgggacccccgggcagagggtcaccatctcttgttctggaagcggctccaacatcagaagtaatggtgtaacttggtaccagcagttcccaggaacggcccccaaactcctcatttatgataataatcagcggccctcaggggtccctggccgattctctggctccaagtctggcacctcaggctccctggccatcagtggactccagtctgaggatgaggctgattattactgtgcagcatgggatgacaacctgaatgcttgggtgttcggcgcagggacccagctgaccgtccta

SEQ ID NO: 174 - DNA, 330 bp, Homo sapiens
  source 1..330: mol_type = other DNA
cagtctgtgctgacgcagccaccctcggtgtctggagcccccagggagagggtcaccatctcctgttctggaagcacctccaatatcgaaaataatgctgtcaactggtaccagcagctcccaggcaaggctcccaaactcctcatctattatgatgatctagtgccctcaggggtctctgaccgattctctggttccaagtccggcacctcagcctccctggccatcagtgggctccagtctggtgatgaggcttattattactgtgcagtttgggatgacagcctgaatggtccggttttcggcggagggaccaaggtcaccgtccta

SEQ ID NO: 175 - DNA, 330 bp, Homo sapiens
  source 1..330: mol_type = other DNA
cagtctgtgctgactcagccaccctcagcgtctgggacccccgggcagagggtcaccatctcttgttctggaagcagctccaacatcggaagtaatactgtaaactggtaccagcagttcccaggaacggcccccaaactcctcatctatagtaataatcagcggccctcaggggtccctgaccgattctctggctccaagtctggcacctcagcctccctggccatcagtgggctccagtctgaggatgaggctgattattactgtgcagcatgggatgacagcctgaatggtcatgtcttcggaactgggaccaaggtcaccgtccta

SEQ ID NO: 176 - DNA, 330 bp, Homo sapiens
  source 1..330: mol_type = other DNA
cagcctgtgctgactcagccaccctcggtgtctgaggcccccaggcagagggtcaccatctcctgttctggaagcagctccaacatcggaaagaatgctgtaaactggtatcagcaattcccaggaaaggctcccaaactcctcatctattatgatgatctactgccctcgggggtctctgaccgattctctggctccaggtctggcacctcagcctccctggccatcagtggactccagtctgaggatgaggctgattattactgtgcagcatgggatgacagactgaaaggatacgtcttcggaactgggacccagctgaccgtccta

SEQ ID NO: 177 - DNA, 333 bp, Homo sapiens
  source 1..333: mol_type = other DNA
caggctgtggtgactcagcctgcctccgtgtctgggtctcctggacagtcgatcaccatctcctgcactggaaccagcagtgacgttggtggttataactatgtctcctggtaccaacagcacccaggcaaagcccccaaactcatgatttatgatgtcagtaatcggccctcaggggtttctaatcgcttctctggctccaagtctggcaacacggcctccctgaccatctctgggctccaggctgaggacgaggctgattattactgcagctcatatacaagcagcagcaccccttatgtcttcggaactgggaccaaggtcaccgtccta

SEQ ID NO: 178 - DNA, 330 bp, Homo sapiens
  source 1..330: mol_type = other DNA
cagtctgtgctgactcagccaccctcggtgtctgaagcccccaggcagagggtcaccatctcctgttctggaagcagctccaatatcgcaaataatgctgtaaactggtaccagcagctcccaggaaaggctcccaaactcctcatttattatgatgatctgttggcctcaggggtctctgaccgattttctggctccaggtctggcacctcagccgccctggccatcagtggcctccagtctgaggatgaggctgattattactgtgcaacatgggatgacgacctgaatggtccgatattcggcggagggaccaaggtcaccgtccta

SEQ ID NO: 179 - DNA, 330 bp, Homo sapiens
  source 1..330: mol_type = other DNA
caggctgtggtgactcagccaccctcagcgtctgggacccccgggcagaggatcaccatctcttgttctggaagcagctccaacatcggaaaaaatattgtaaactggtaccagcaggtcccaggagcggcccccaaactcctcatctataataatagtcagcggccctcaggggtccctgaccgattctctggctccaagtctggcacctcagcctccctggccatcagtgggctccagtctgaggatgaggctgattattactgtgcagcatgggatgacagcctgagatcttggctgttcggcggagggaccaaggtcaccgtccta

SEQ ID NO: 180 - DNA, 333 bp, Homo sapiens
  source 1..333: mol_type = other DNA
cagcctgtgctgactcagcctgcctccgtgtctgggtctcctggacagtcgatcaccatctcctgcactggaaccagcagtgacgttggtggttataactatgtctcctggtaccaacagcacccaggcaaagcccccaaactcatgatttatgaggtcagtaatcggccctcaggggtttctaatcgcttctctggctccaagtctggcaacacggcctccctgaccatctctgggctccaggctgaggacgaggctgattattactgcagctcatatacaagcagcagcactccttgggtgttcggcggagggaccaaggtcaccgtccta

SEQ ID NO: 181 - DNA, 333 bp, Homo sapiens
  source 1..333: mol_type = other DNA
caggctgtggtgactcagcctgcctccgtgtctgggtctcctggacagtcgatcaccatctcctgcactggaaccagcagtgacgttggtggttataactatgtctcctggtaccaacagcacccaggcaaagcccccaaactcatgatttatgaggtcactaatcggccctcaggggtttctaatcgcttctctggctccaagtctggcaacacggcctccctgaccatctctgggctccaggctgaggacgaggctgattattactgcagctcatatacaagcagcagcaccccgtatgtcttcggaactgggaccaaggtcaccgtccta

SEQ ID NO: 182 - DNA, 321 bp, Homo sapiens
  source 1..321: mol_type = other DNA
gacatccagatgacccagtctccatcctccctgtctgcatctgtaggagacagagtcaccatcacttgccaggcgagtcaggacattagcaactatttaaattggtatcagcagaaaccagggaaagcccctaagctcctgatctacgatgcatccaatttggaaacaggggtcccatcaaggttcagtggaagtggatctgggacagattttactttcaccatcagcagcctgcagcctgaagatattgcaacatattactgtcaacagtatgataatctccccctcactttcggcggagggaccaaagtggatatcaaa

(19) United States
(12) Patent Application Publication
(10) Pub. No.: US 2024/0392001 A1
(43) Pub. Date: Nov. 28, 2024

(54) LILRB2-SPECIFIC MONOCLONAL ANTIBODIES AND METHODS OF THEIR USE

(71) Applicant: THE BOARD OF REGENTS OF THE UNIVERSITY OF TEXAS SYSTEM, Austin, TX (US)

(72) Inventors: Zhiqiang AN, Houston, TX (US); Ningyan ZHANG, Houston, TX (US); Peng ZHAO, Houston, TX (US)

(21) Appl. No.: 18/691,249

(22) PCT Filed: Sep. 13, 2022
(86) PCT No.: PCT/US2022/076383

§ 371 Date: Mar. 12, 2024

Related U.S. Application Data
(60) Provisional application No. 63/243,472, filed on Sep. 13, 2021.

(51) Int. Cl. C07K 16/28

(52) U.S. Cl. CPC C07K 16/2803; C07K 2317/33; C07K 2317/92

(57) ABSTRACT

Isolated or recombinant anti-LILRB2 monoclonal antibodies are provided. In some embodiments, the antibodies herein can be used for the detection, diagnosis and/or therapeutic treatment of human diseases, such as Alzheimer's Disease. In further aspects, the LILRB2-binding antibodies can affect cellular signaling mediated through at least the oA and PS-mediated TREM2 and LILRB2 co-ligation signaling pathway and can be used to modulate microglia function.

### REFERENCE TO A SEQUENCE LISTING SUBMITTED VIA ELECTRONIC FILING

The content of the XML file of the sequence listing named “UTH-013PCT0_sequence_listing_ST26_FILED.XML” which is 164 KB in size was created on Sep. 11, 2022, and electronically submitted to the USPTO's Patent Center herewith the present application is incorporated by reference in its entirety.

### CROSS-REFERENCE TO PRIOR APPLICATIONS

This application claims the benefit of U.S. Provisional Appl. No. 63/243,472, filed Sep. 13, 2021. The content of the foregoing application is relied upon and is incorporated by reference herein in its entirety.

### BACKGROUND

As discussed in Zhao et al. (“TREM2 Is a Receptor for-Amyloid that Mediates Microglial Function”: 2018; Neuron 97:1023-1031: which is incorporated herein by reference in its entirety), Alzheimer's disease (AD) is an age-dependent neurodegenerative disorder characterized by senile plaques enriched with β-amyloid (Aβ) peptides and neurofibrillary tangles comprised of hyperphosphorylated tau. Oligomeric forms of AB (oAβ) are believed to trigger a pathological cascade leading to AD. Recent genome-wide association studies have identified numerous genetic loci altered in AD, which may not impact directly on Aβ generation or tau aggregation. Interestingly, a subset of these genes, including TREM2 (Triggering Receptor Expressed on Myeloid cells 2), is expressed in myeloid cell types such as microglia, suggesting that immune cell dysfunction may play a key role in AD pathogenesis. In addition to familial β-amyloid precursor protein (APP), presenilin (PS) 1/2 alleles linked to early AD onset, variations in ApoE (ε4), and the TREM2 ectodomain (R47H) are currently associated with the highest risk of AD. Additional variants of the TREM2 ectodomain—e.g., R62H—are also linked to increased AD risk. TREM2 has been shown to mediate phagocytic clearance of apoptotic cell debris and modulate inflammatory response. TREM2 binds to its adaptor, DNAX-activating protein of 12 kDa (DAP12) on the surface of microglia to enact innate immune responses and downstream cellular responses or signaling pathways. Despite recent identification of lipids and ApoE as TREM2 ligands, biological ligands for TREM2 and their consequent impact on microglial function in AD pathogenesis remain unclear.

There remains a need for diagnostic and therapeutic compositions and methods to detect, prevent and treat pathological processes that occur during the development of AD and among others, additional forms of dementia.

### SUMMARY

Monoclonal antibody compositions are now provided herein which may be used in diagnosis and/or treatment of a neurologic or neurodegenerative disease and disorder including, but, not limited to, Alzheimer's Disease (AD), Parkinson's Disease (PD, dementia, dementia with Lewy bodies (DLB) and others, including neuroinflammatory processes and those involving microglia.

Advantageously, this discovery provides its user with means to treat certain types of neurodegenerative diseases and dementia that have failed to respond adequately to other conventional treatments.

Described herein are monoclonal antibodies that bind to LILRB2. In further aspects, the provided LILRB2-binding antibodies can affect cellular signaling mediated through at least the oAβ and PS-mediated TREM2 and Leukocyte Immunoglobulin-like Receptor subfamily B member 2 (“LILRB2”) co-ligation signaling pathway and can be used to modulate microglia function.

Thus, in one aspect, there is provided an isolated or recombinant monoclonal antibody that specifically binds to LILRB2. In certain aspects, an antibody that competes for the binding of LILRB2 with the NeuB2-8, NeuB2-9, NeuB2-13, NeuB2-19, NeuB2-29, NeuB2-37, NeuB2-40, NeuB2-55, NeuB2-60, NeuB2-80, NeuB2-93, NeuB2-110, or NeuB2-128 monoclonal antibody is provided. In certain aspects, the antibody may comprise all or part of the heavy chain variable region and/or light chain variable region of the NeuB2-8, NeuB2-9, NeuB2-13, NeuB2-19, NeuB2-29, NeuB2-37, NeuB2-40, NeuB2-55, NeuB2-60, NeuB2-80, NeuB2-93, NeuB2-110, or NeuB2-128 monoclonal antibodies.

In a further aspect, the antibody may comprise an amino acid sequence that corresponds to a first, second, and/or third complementarity determining region (CDR) from the light variable and/or heavy variable chain of the NeuB2-8, NeuB2-9, NeuB2-13, NeuB2-19, NeuB2-29, NeuB2-37, NeuB2-40, NeuB2-55, NeuB2-60, NeuB2-80, NeuB2-93, NeuB2-110, or NeuB2-128 monoclonal antibodies of the present embodiments.

In certain embodiments, the isolated antibody comprises CDR sequences at least 80%, 85%, 90%, 91%, 92%, 93%, 94%, 95%, 96%, 97%, 98%, 99% or 100% identical to the CDR regions of the NeuB2-8, NeuB2-9, NeuB2-13, NeuB2-19, NeuB2-29, NeuB2-37, NeuB2-40, NeuB2-55, NeuB2-60, NeuB2-80, NeuB2-93, NeuB2-110, or NeuB2-128 heavy and light chain amino acid sequences. In further aspects, an antibody comprises CDR regions identical to the NeuB2-8, NeuB2-9, NeuB2-13, NeuB2-19, NeuB2-29, NeuB2-37, NeuB2-40, NeuB2-55, NeuB2-60, NeuB2-80, NeuB2-93, NeuB2-110, or NeuB2-128 CDR regions, except for one or two amino acid substitutions, deletions, or insertions at one or more of the CDRs.

In yet another aspect, the present disclosure provides a composition comprising the antibody or recombinant polypeptide of any one of the above embodiments.

In yet another aspect, the present disclosure provides a host cell comprising one or more polynucleotide molecule(s) encoding an antibody or a recombinant polypeptide of any one of the above embodiments.

The numbers, E5, E6, and E7 and the like are used interchangeably herein with 10⁵, 10⁶, and 10⁷, respectively, and the like.

The term “comprising” and variations thereof (e.g., comprises, includes, etc.) do not have a limiting meaning where these terms appear in the description and claims.

As used herein, “a”, “an”, “the,” “at least one,” and “one or more” are used interchangeably, unless the context clearly dictates otherwise.

Also herein, the recitations of numerical ranges by endpoints include all numbers subsumed within that range (e.g., 500 to 7000 nm includes 500, 530, 551, 575, 583, 592, 600, 620, 650, 700, etc.).

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the invention.

An “agonist” antibody or an “activating” antibody is an antibody that induces (e.g., increases) one or more activities or functions of the antigen after the antibody binds the antigen.

An “antagonist” antibody or a “blocking” antibody is an antibody that reduces or eliminates (e.g., decreases) antigen binding to one or more ligand after the antibody binds the antigen, and/or that reduces or eliminates (e.g., decreases) one or more activities or functions of the antigen after the antibody binds the antigen. In some embodiments, antagonist antibodies, or blocking antibodies substantially or completely inhibit antigen binding to one or more ligand and/or one or more activities or functions of the antigen.

As used herein, “percent (%) amino acid sequence identity” and “homology” with respect to a peptide, polypeptide or antibody sequence refers to the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms known in the art needed to achieve maximal alignment over the full-length of the sequences being compared.

A “host cell” includes an individual cell or cell culture that can be or has been a recipient for vector(s) for incorporation of polynucleotide inserts. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) of this invention.

The term “isolated molecule” (where the molecule is, for example, a polypeptide, a polynucleotide, or an antibody) is a molecule that by virtue of its origin or source of derivation (1) is not associated with naturally associated components that accompany it in its native state, (2) is substantially free of other molecules from the same species (3) is expressed by a cell from a different species, or (4) does not occur in nature. Accordingly, an anti-LILRB2 antibody of the present disclosure, is a nucleic acid molecule that is identified and separated from at least one contaminant nucleic acid molecule with which it is ordinarily associated in the environment in which it was produced. Preferably, the isolated nucleic acid is free of association with all components associated with the production environment. The isolated nucleic acid molecules encoding the polypeptides and antibodies herein is in a form other than in the form or setting in which it is found in nature. Isolated nucleic acid molecules therefore are distinguished from nucleic acid encoding the polypeptides and antibodies herein existing naturally in cells. Moreover, a molecule that is chemically synthesized, or expressed in a cellular system different from the cell from which it naturally originates, will be “isolated” from its naturally associated components. A molecule also may be rendered substantially free of naturally associated components by isolation, using purification techniques well known in the art. Molecule purity or homogeneity may be assayed by a number of means well known in the art. For example, the purity of a polypeptide sample may be assayed using polyacrylamide gel electrophoresis and staining of the gel to visualize the polypeptide using techniques well known in the art. For certain purposes, higher resolution may be provided by using HPLC or other means well known in the art for purification.

The term “epitope” refers to that portion of a molecule capable of being recognized by and bound by an antibody molecule, or antigen-binding portion thereof, at one or more of the antibody molecule's antigen-binding regions. Epitopes can consist of defined regions of primary secondary or tertiary protein structure and includes combinations of secondary structural units or structural domains of the target recognized by the antigen binding regions of the antibody, or antigen-binding portion thereof. Epitopes can likewise consist of a defined chemically active surface grouping of molecules such as amino acids or sugar side chains and have specific three-dimensional structural characteristics as well as specific charge characteristics. The term “antigenic epitope” as used herein, is defined as a portion of a polypeptide to which an antibody molecule can specifically bind as determined by any method well known in the art, for example, by conventional immunoassays, antibody competitive binding assays or by x-ray crystallography or related structural determination methods (for example NMR).

The term “binding affinity” or “K_{D}” refers to the dissociation rate of a particular antigen-antibody interaction. The K_{D} is the ratio of the rate of dissociation, also called the “off-rate (k_{off})”, to the association rate, or “on-rate (kₒₙ)”. Thus, K_{D} equals k_{off}/kₒₙ and is expressed as a molar concentration (M). It follows that the smaller the K_{D}, the stronger the affinity of binding. Therefore, a K_{D} of 1 μM indicates weak binding affinity compared to a K_{D} of 1 nM. K_{D} values for antibodies can be determined using methods well established in the art. One method for determining the K_{D} of an antibody is by using surface plasmon resonance (SPR), typically using a biosensor system such as a Biacore® system.

The phrase “effective amount” or “therapeutically effective amount” as used herein refers to an amount necessary (at dosages and for periods of time and for the means of administration) to achieve the desired therapeutic result. An effective amount is at least the minimal amount, but less than a toxic amount, of an active agent which is necessary to impart therapeutic benefit to a subject.

The term “inhibit” or “neutralize” as used herein with respect to bioactivity of an antibody molecule of the invention means the ability of the antibody to substantially antagonize, prohibit, prevent, restrain, slow, disrupt, eliminate, stop, reduce or reverse for example progression or severity of that which is being inhibited including, but not limited to, a biological activity or binding interaction of the antibody molecule to LILRB2.

As used herein, “vector” means a construct, which is capable of delivering, and, preferably, expressing, one or more gene(s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells.

The term “treating”, as used herein, unless otherwise indicated, means reversing, alleviating, inhibiting the progress of, delaying the progression of, delaying the onset of, or preventing the disorder or condition to which such term applies, or one or more symptoms of such disorder or condition. The term “treatment”, as used herein, unless otherwise indicated, refers to the act of treating as defined above. The term “treating” also includes adjuvant and neoadjuvant treatment of a subject. For the avoidance of doubt, reference herein to “treatment” includes reference to curative, palliative and prophylactic treatment. For the avoidance of doubt, references herein to “treatment” also include references to curative, palliative and prophylactic treatment.

The term “comprising” and variations thereof (e.g., comprises, includes, etc.) do not have a limiting meaning where these terms appear in the description and claims.

All scientific and technical terms used herein have meanings commonly used in the art unless otherwise specified. The definitions provided herein are to facilitate understanding of certain terms used frequently in this application and are not meant to exclude a reasonable interpretation of those terms in the context of the present disclosure.

It is understood that aspect and embodiments of the present disclosure described herein include “comprising,” “consisting,” and “consisting essentially of” aspects and embodiments.

Unless otherwise indicated, all numbers in the description and the claims expressing feature sizes, amounts, and physical properties used in the specification and claims are to be understood as being modified in all instances by the term “about.” Accordingly, unless indicated to the contrary, the numerical parameters set forth in the foregoing specification and attached claims are approximations that can vary depending upon the desired properties sought to be obtained by those skilled in the art utilizing the teachings disclosed herein. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviations found in their respective testing measurements.

The above summary of the present invention is not intended to describe each disclosed embodiment or every implementation of the present invention. The description that follows more particularly exemplifies illustrative embodiments.

### BRIEF DESCRIPTION OF THE DRAWINGS

FIG. 1 is a graph showing purified NeuB2-mAbs bind to recombinantly produced LILRB2 extracellular domain (ECD) protein in an ELISA assay. The EC₅₀ values were determined from the concentration titration curves.

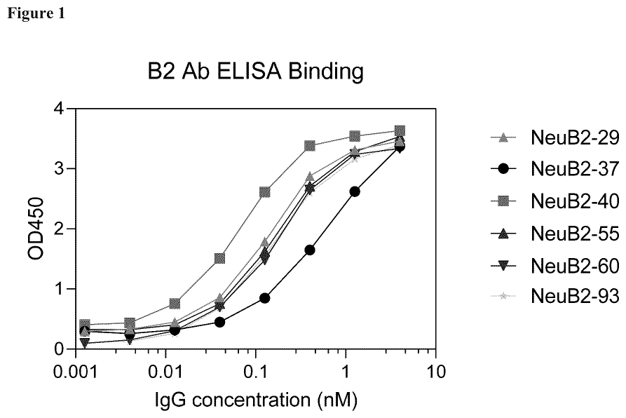

FIG. 2 is a graph showing no cross-reactivity of NeuB2-mAbs to other LILRB and LILRA family members.

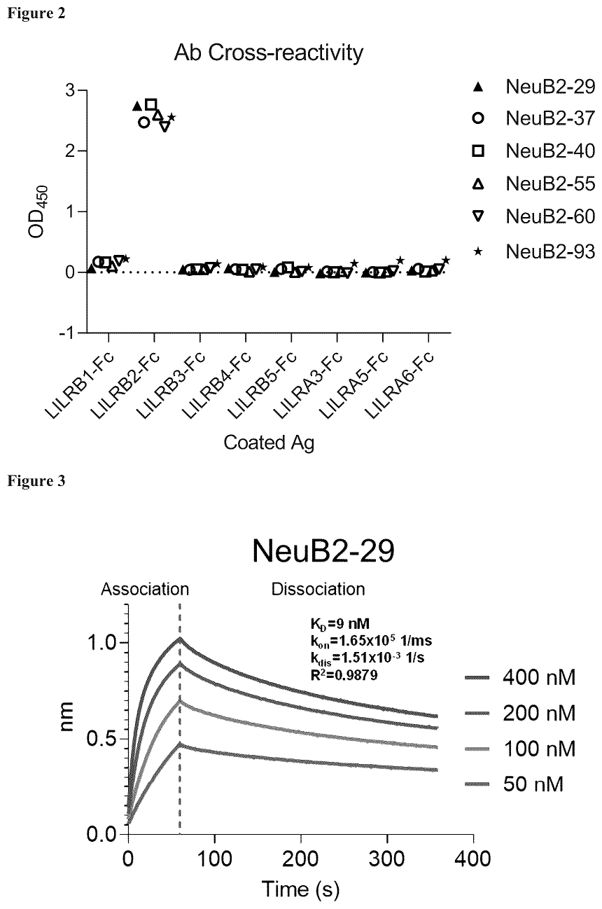

FIG. 3 is a graph showing the binding kinetics of NeuB2 mAbs using BLI based Octet 96-Red instrument.

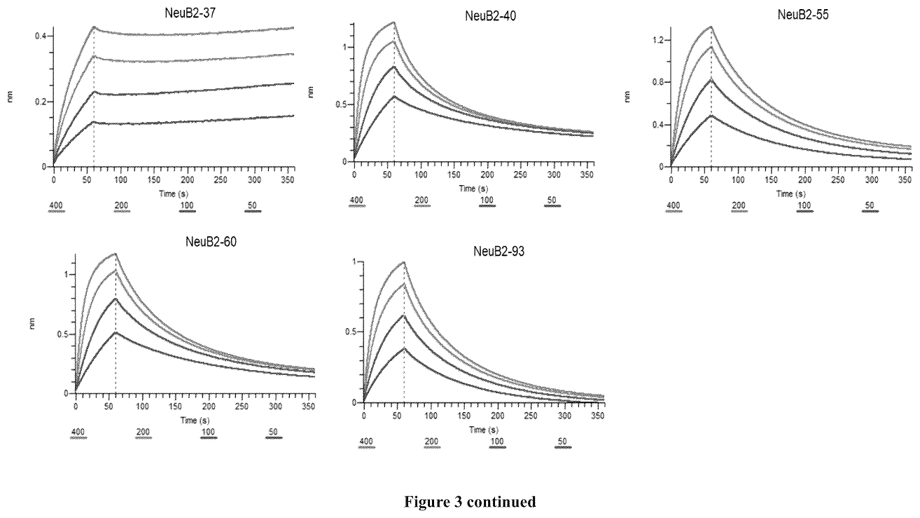

FIG. 4 is a graph showing that monoclonal antibody NewuB2-29 binds to HEK293T cells that express LILRB2 on their surface, as measured in a flow cytometry assay.

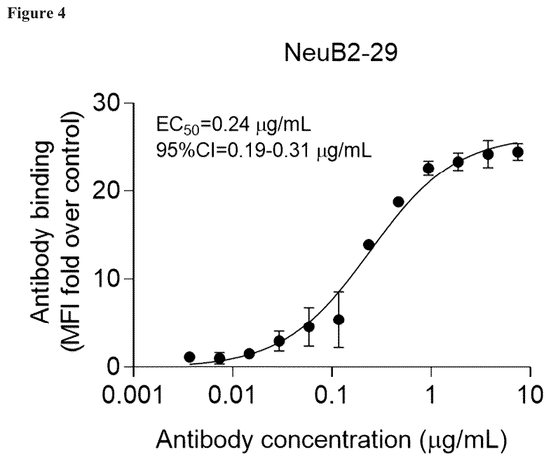

FIG. 5A provides graphs showing purified NeuB2-29 (Ab29) blocked LILRB2 interactions with ligands PS and oAβ, as measured using an Octet 96-Red instrument.

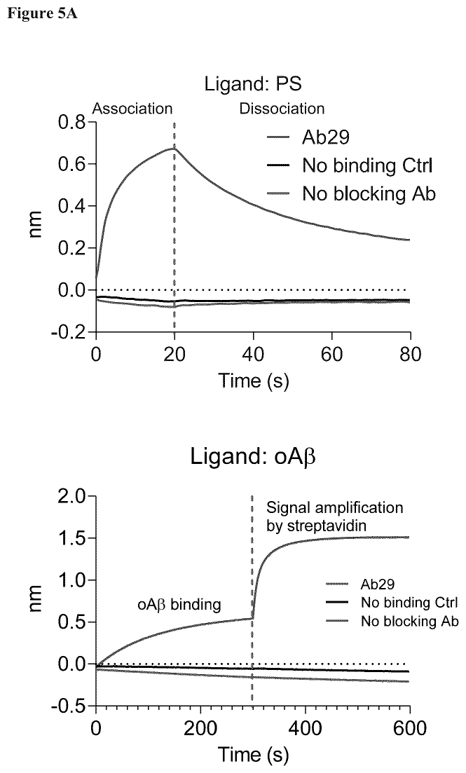

FIG. 5B provides graphs showing purified NeuB2-29 (Ab29) blocked LILRB2 interactions with ligands PS and oAβ, as measured using a LILRB2-chimeric cell reporter assay.

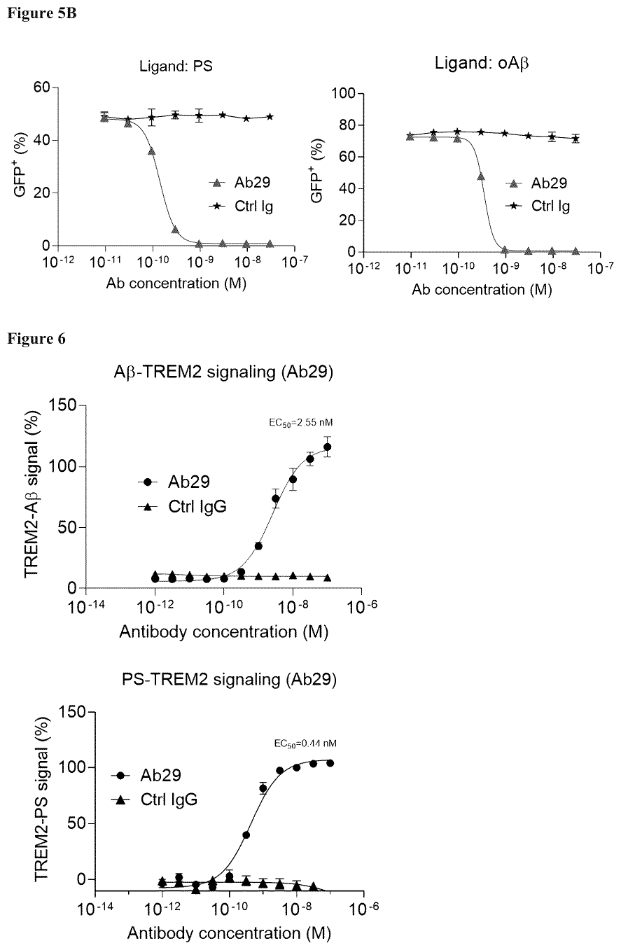

FIG. 6 provides graphs showing NeuB2-29 mAb (Ab29) rescued ligand induced TREM2 signaling, as measured using the reporter cell assay.

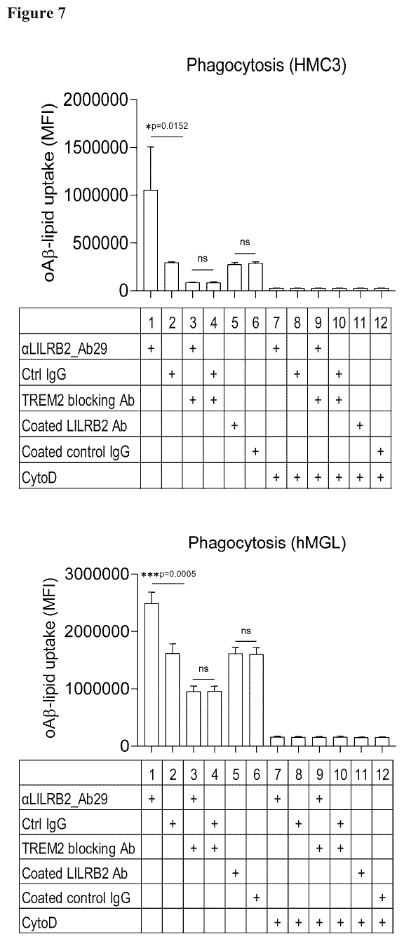

FIG. 7 provides graphs showing that NeuB2-9 (Ab29) mAb increased microglia phagocytosis of oAβ complex and that the activity depended on TREM2 ligation with the ligand.

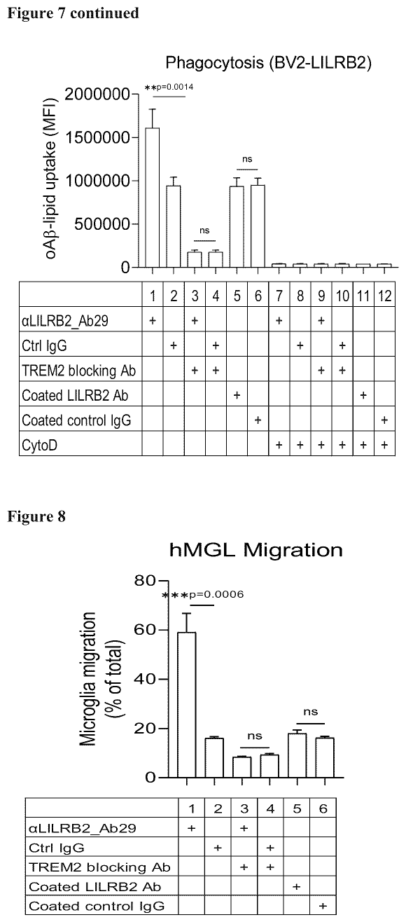

FIG. 8 is a graph showing that NeuB2-29 (Ab29) mAb promoted migration of human microglia cells mediated through TREM2 signaling.

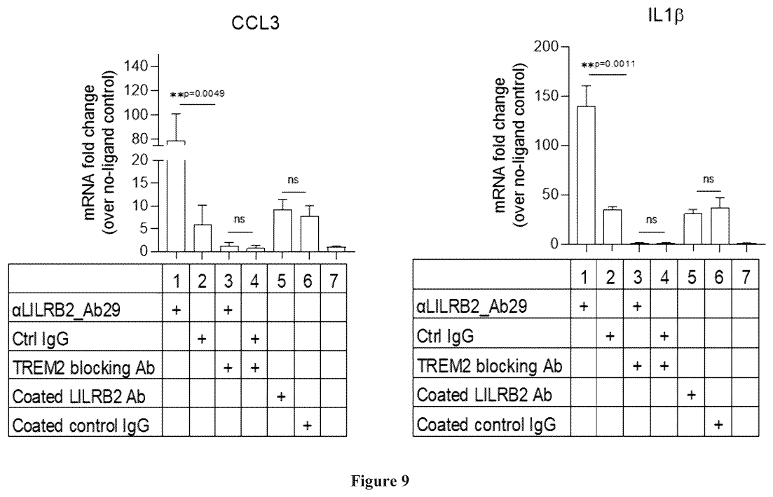

FIG. 9 provides graphs showing that NeuB2-29 (Ab29) increased gene transcription of a panel of inflammatory cytokine/chemokines in hMGL cells mediated through TREM2 signaling.

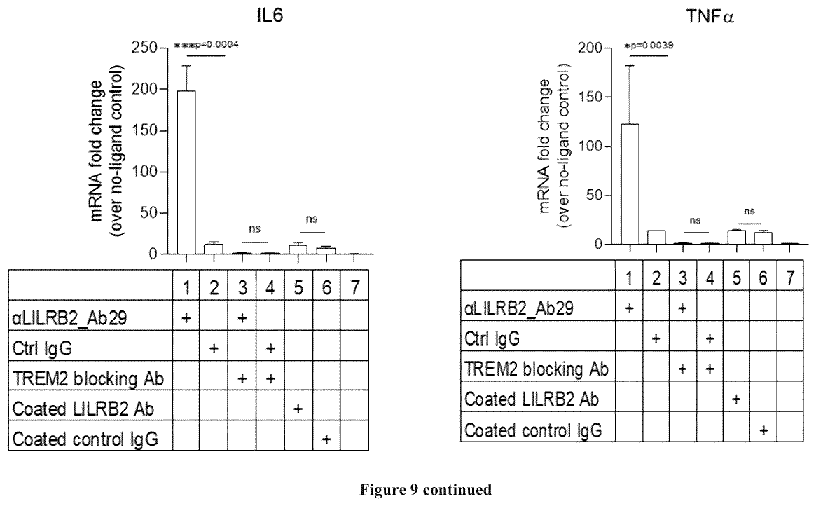

### DETAILED DESCRIPTION

The present disclosure describes a panel of monoclonal antibodies, and fragments thereof, with binding affinity to LILRB2. The antibodies bind specifically to LILRB2 and block interactions between LILRB2 with ligands oAβ and PS without cross-reactivity to receptors in the LILRB and LILRA families (Zhao et al., Molecular Neurodegeneration, 17:44 2022). The antibodies of the present disclosure can be used to treat diseases of the brain such as neurodegenerative diseases and disorders including, but, not limited to, Alzheimer's Disease (AD), Parkinson's Disease (PD), dementia, dementia with Lewy bodies (DLB) and others, including neuroinflammatory processes and those involving microglia, for example.

### Antibodies of the Embodiments

In certain embodiments, an antibody or a fragment thereof that binds to at least a portion of LILRB2 protein and inhibits LILRB2 signaling are contemplated. As used herein, the term “antibody” is intended to refer broadly to any immunologic binding agent, such as IgG, IgM, IgA, IgD, IgE, and genetically modified IgG as well as polypeptides comprising antibody CDR domains that retain antigen binding activity. The antibody may be selected from the group consisting of a chimeric antibody, an affinity matured antibody, a polyclonal antibody, a monoclonal antibody, a humanized antibody, a human antibody, or an antigen-binding antibody fragment or a natural or synthetic ligand. Preferably, the anti-LILRB2 antibody is a monoclonal antibody or a humanized antibody.

An “antibody molecule” encompasses an antibody of any class, such as IgG, IgA, or IgM (or sub-class thereof), and the antibody need not be of any particular class. Depending on the antibody amino acid sequence of the constant region of its heavy chains, immunoglobulins can be assigned to different classes. There are five major classes of immunoglobulins: IgA, IgD, IgE, IgG, and IgM, and several of these may be further divided into subclasses (isotypes), for example IgG1, IgG2, IgG3, IgG4, IgA1 and IgA2. The heavy-chain constant regions that correspond to the different classes of immunoglobulins are called alpha, delta, epsilon, gamma, and mu, respectively. The subunit structures and three-dimensional configurations of different classes of immunoglobulins are well known.

The term “antigen binding portion” of an antibody molecule, as used herein, refers to one or more fragments of an intact antibody that retain the ability to specifically bind to LILRB2. Antigen binding functions of an antibody molecule can be performed by fragments of an intact antibody. Examples of binding fragments encompassed within the term “antigen binding portion” of an antibody molecule include Fab; Fab′; F(ab′)2; an Fd fragment consisting of the V_{H} and CH1 domains; an Fv fragment consisting of the V_{L} and V_{H} domains of a single arm of an antibody; a single domain antibody (dAb) fragment, and an isolated complementarity determining region (CDR).

The term “Fc region” is used to define a C-terminal region of an immunoglobulin heavy chain. The “Fc region” may be a native sequence Fc region or a variant Fc region. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The numbering of the residues in the Fc region is that of the EU index as in Kabat. The Fc region of an immunoglobulin generally comprises two constant domains, CH2 and CH3. As is known in the art, an Fc region can be present in dimer or monomeric form.

A “variable region” of an antibody refers to the variable region of the antibody light chain or the variable region of the antibody heavy chain, either alone or in combination. As known in the art, the variable regions of the heavy and light chain each consist of four framework regions (FRs) connected by three complementarity determining regions (CDRs) also known as hypervariable regions, contribute to the formation of the antigen binding site of antibodies. When choosing FR to flank CDRs, for example when humanizing or optimizing an antibody, FRs from antibodies which contain CDR sequences in the same canonical class are preferred.

As used herein the term “conservative substitution” refers to replacement of an amino acid with another amino acid which does not significantly deleteriously change the functional activity. A preferred example of a “conservative substitution” is the replacement of one amino acid with another amino acid which has a value greater than 0 in a BLOSUM 62 substitution matrix (see Henikoff & Henikoff, 1992, PNAS 89:10915-10919).

Thus, by known means and as described herein, polyclonal or monoclonal antibodies, antibody fragments, and binding domains and CDRs (including engineered forms of any of the foregoing) may be created that are specific to LILRB2 protein, one or more of its respective epitopes, or conjugates of any of the foregoing, whether such antigens or epitopes are isolated from natural sources or are synthetic derivatives or variants of the natural compounds.

Examples of antibody fragments suitable for the present embodiments include, without limitation: (i) the Fab fragment, consisting of V_{L}, V_{H}, CL, and CH1 domains; (ii) the “Fd” fragment consisting of the V_{H} and CH1 domains; (iii) the “Fv” fragment consisting of the V_{L} and V_{H} domains of a single antibody; (iv) the “dAb” fragment, which consists of a V_{H} domain; (v) isolated CDR regions; (vi) F(ab′)2 fragments, a bivalent fragment comprising two linked Fab fragments; (vii) single chain Fv molecules (“scFv”), wherein a V_{H} domain and a V_{L} domain are linked by a peptide linker that allows the two domains to associate to form a binding domain; (viii) bi-specific single chain Fv dimers (see, for example, U.S. Pat. No. 5,091,513); and (ix) diabodies, multivalent or multispecific fragments constructed by gene fusion (see, for example, US Patent App. Pub. No. 20050214860). Fv, scFv, or diabody molecules may be stabilized by the incorporation of disulphide bridges linking the V_{H} and V_{L} domains. Minibodies comprising a scFv joined to a CH3 domain may also be made (See, for example, Hu et al. 1996, “Minibody: A Novel Engineered Anti-Carcinoembryonic Antigen Antibody Fragment (Single-Chain Fv-CH3) Which Exhibits Rapid, High-Level Targeting of Xenografts”, Cancer Res. 56:3055-3061).

Antibody-like binding peptidomimetics are also contemplated in embodiments. Liu et al. (Murali, R.; Liu. Q.; Cheng, X.; Berezov, A.; Richter. M.; Furuchi, K.; Greene, M. I.; Zhang, H. Antibody like peptidomimetics as large scale immunodetection probes. Cell. Mol. Biol. (Noisy-le-grand) 2003. 49:209-216, which is incorporated herein by reference in its entirety) describe “antibody like binding peptidomimetics” (ABiPs), which are peptides that act as pared-down antibodies and have certain advantages of longer serum half-life as well as less cumbersome synthesis methods.

A monoclonal antibody (or “MAb”) is a single species of antibody wherein every antibody molecule recognizes the same epitope because all antibody producing cells are derived from a single B-lymphocyte cell line. The methods for generating monoclonal antibodies (MAbs) generally begin along the same lines as those for preparing polyclonal antibodies. In some embodiments, rodents such as mice and rats are used in generating monoclonal antibodies. In some embodiments, rabbit, sheep, or frog cells are used in generating monoclonal antibodies. The use of rats is well known and may provide certain advantages. Mice (e.g., BALB/c mice) are routinely used and generally give a high percentage of stable fusions.

Hybridoma technology involves the fusion of a single B lymphocyte from a mouse previously immunized, for example, with a LILRB2 antigen with an immortal myeloma cell (usually mouse myeloma). This technology provides a method to propagate a single antibody producing cell for an indefinite number of generations, such that unlimited quantities of structurally identical antibodies having the same antigen or epitope specificity (monoclonal antibodies) may be produced.

Plasma B cells (CD45+CD5−CD19+) may be isolated from freshly prepared rabbit peripheral blood mononuclear cells of immunized rabbits and further selected for LILRB2 binding cells. After enrichment of antibody producing B cells, total RNA may be isolated and cDNA synthesized. DNA sequences of antibody variable regions from both heavy chains and light chains may be amplified, constructed into a phage display Fab expression vector, and transformed into E. coli. LILRB2 specific binding Fab may be selected out through multiple rounds enrichment panning and sequenced. Selected LILRB2 binding hits may be expressed as full-length IgG in rabbit and rabbit/human chimeric forms using a mammalian expression vector system in human embryonic kidney (HEK293) cells (Invitrogen) and purified using a protein G resin with a fast protein liquid chromatography (FPLC) separation unit.

In one embodiment, the antibody is a chimeric antibody, for example, an antibody comprising antigen binding sequences from a non-human donor grafted to a heterologous non-human, human, or humanized sequence (e.g., framework and/or constant domain sequences). Methods have been developed to replace light and heavy chain constant domains of the monoclonal antibody with analogous domains of human origin, leaving the variable regions of the foreign antibody intact. Alternatively, “fully human” monoclonal antibodies are produced in mice transgenic for human immunoglobulin genes. Methods have also been developed to convert variable domains of monoclonal antibodies to more human form by recombinantly constructing antibody variable domains having both rodent, for example, mouse, and human amino acid sequences. In “humanized” monoclonal antibodies, only the hypervariable CDR is derived from mouse monoclonal antibodies, and the framework and constant regions are derived from human amino acid sequences (see, for example, U.S. Pat. Nos. 5,091,513 and 6,881,557, which are incorporated herein by reference in their entirety). It is thought that replacing amino acid sequences in the antibody that are characteristic of rodents with amino acid sequences found in the corresponding position of human antibodies will reduce the likelihood of adverse immune reaction during therapeutic use. A hybridoma or other cell producing an antibody may also be subject to genetic mutation or other changes, which may or may not alter the binding specificity of antibodies produced by the hybridoma.

Methods for producing polyclonal antibodies in various animal species, as well as for producing monoclonal antibodies of various types, including humanized, chimeric, and fully human, are well known in the art and highly predictable. For example, the following U.S. patents and patent applications, which are incorporated herein by reference in their entirety, provide enabling descriptions of such methods: U.S. Patent Application Nos. 2004/0126828 and 2002/0172677; and U.S. Pat. Nos. 3,817,837; 3,850,752; 3,939,350; 3,996,345; 4,196,265; 4,275,149; 4,277,437; 4,366,241; 4,469,797; 4,472,509; 4,606,855; 4,703,003; 4,742,159; 4,767,720; 4,816,567; 4,867,973; 4,938,948; 4,946,778; 5,021,236; 5,164,296; 5,196,066; 5,223,409; 5,403,484; 5,420,253; 5,565,332; 5,571,698; 5,627,052; 5,656,434; 5,770,376; 5,789,208; 5,821,337; 5,844,091; 5,858,657; 5,861,155; 5,871,907; 5,969,108; 6,054,297; 6,165,464; 6,365,157; 6,406,867; 6,709,659; 6,709,873; 6,753,407; 6,814,965; 6,849,259; 6,861,572; 6,875,434; and 6,891,024.

Antibodies may be produced from any animal source, including birds and mammals. Preferably, the antibodies are ovine, murine (e.g., mouse and rat), rabbit, goat, guinea pig, camel, horse, or chicken. In addition, newer technology permits the development of and screening for human antibodies from human combinatorial antibody libraries. For example, bacteriophage antibody expression technology allows specific antibodies to be produced in the absence of animal immunization, as described in U.S. Pat. No. 6,946,546, which is incorporated herein by reference.

It is fully expected that antibodies to LILRB2 will have the ability to modulate, by binding to LILRB2, human microglia activity regardless of the source (e.g., animal species, monoclonal cell line, or other source) of the antibody. Certain animal species may be less preferable for generating therapeutic antibodies because they may be more likely to cause allergic response due to activation of the complement system through the “Fc” portion of the antibody. However, whole antibodies may be enzymatically digested into “Fc” (complement binding) fragment, and into antibody fragments having the binding domain or CDR. Removal of the Fc portion reduces the likelihood that the antigen antibody fragment will elicit an undesirable immunological response, and thus, antibodies without Fc may be preferential for prophylactic or therapeutic treatments. As described above, antibodies may also be constructed so as to be chimeric or partially or fully human, so as to reduce or eliminate the adverse immunological consequences resulting from administering to an animal an antibody that has been produced in, or has sequences from, other species.

Substitutional variants typically contain the exchange of one amino acid for another at one or more sites within the monoclonal antibody protein and may be designed to modulate one or more properties of the polypeptide, with or without the loss of other functions or properties. Substitutions may be conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Alternatively, substitutions may be non-conservative such that a function or activity of the polypeptide is affected. Non-conservative changes typically involve substituting a residue with one that is chemically dissimilar, such as a polar or charged amino acid for a nonpolar or uncharged amino acid, and vice versa.

Proteins (e.g., monoclonal antibodies) of the present disclosure may be isolated (e.g., enriched and/or purified to some degree) and/or may be recombinant or synthesized in vitro. Alternatively, a nonrecombinant or recombinant protein may be isolated from bacteria. It is also contemplated that a bacteria containing such a variant may be implemented in compositions and methods. Consequently, a protein need not be isolated.

Thus, the present disclosure provides an isolated or recombinant monoclonal antibody that specifically binds to LILRB2. In certain aspects, an antibody that competes for the binding of LILRB2 with the (NeuB2-8), (NeuB2-9), (NeuB2-13), (NeuB2-19), (NeuB2-29), (NeuB2-37), (NeuB2-40), (NeuB2-55), (NeuB2-60), or (NeuB2-80), (NeuB2-8), (NeuB2-93), (NeuB2-110), or (NeuB2-128) monoclonal antibody (each disclosed and described herein) is provided. In certain aspects, the antibody may comprise all or part of the heavy chain variable region and/or light chain variable region of the (NeuB2-8), (NeuB2-9), (NeuB2-13), (NeuB2-19), (NeuB2-29), (NeuB2-37), (NeuB2-40), (NeuB2-55), (NeuB2-60), or (NeuB2-80), (NeuB2-8), (NeuB2-93), (NeuB2-110), or (NeuB2-128) monoclonal antibodies.

It is contemplated that in compositions of the present disclosure there is between about 0.001 mg and about 10 mg of total polypeptide, peptide, and/or protein per ml. Thus, the concentration of protein in a composition can be about, at least about or at most about 0.001, 0.010, 0.050, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0 mg/ml or more (or any range derivable therein). Of this, about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% may be an antibody that binds LILRB2.

An antibody or preferably an immunological portion of an antibody, can be chemically conjugated to, or expressed as, a fusion protein with other proteins. For purposes of this specification and the accompanying claims, all such fused proteins are included in the definition of antibodies or an immunological portion of an antibody.

Embodiments provide antibodies and antibody-like molecules against LILRB2, polypeptides and peptides that are linked to at least one agent to form an antibody conjugate or payload. In order to increase the efficacy of antibody molecules as diagnostic or therapeutic agents, it is conventional to link or covalently bind or complex at least one desired molecule or moiety. Such a molecule or moiety may be, but is not limited to, at least one effector or reporter molecule. Effector molecules comprise molecules having a desired activity, e.g., cytotoxic activity. Non-limiting examples of effector molecules that have been attached to antibodies include toxins, therapeutic enzymes, antibiotics, radio-labeled nucleotides and the like. By contrast, a reporter molecule is defined as any moiety that may be detected using an assay. Non-limiting examples of reporter molecules that have been conjugated to antibodies include enzymes, radiolabels, haptens, fluorescent labels, phosphorescent molecules, chemiluminescent molecules, chromophores, luminescent molecules, photoaffinity molecules, colored particles or ligands, such as biotin.

Several methods are known in the art for the attachment or conjugation of an antibody to its conjugate moiety. Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3α-6α-diphenylglycouril attached to the antibody. Monoclonal antibodies may also be reacted with an enzyme in the presence of a coupling agent such as glutaraldehyde or periodate. Conjugates with fluorescein markers are prepared in the presence of these coupling agents or by reaction with an isothiocyanate.

In another aspect, the present disclosure provides polynucleotides that can be expressed (e.g., transcribed and translated) in a suitable host to produce a LILRB2-binding polypeptides or portions thereof. It is contemplated that such polynucleotide sequences can be synthesized using methods that are known in the art and the polynucleotides can be cloned in a suitable expression vector by means known in the art and the expression vector can be used in vivo or in vitro to express the LILRB2-binding polypeptide encoded by the polynucleotide sequences.

In certain embodiments, a polynucleotide of the present disclosure comprises a portion having at least 80%, 85%, 90%, 91%, 92%, 93%, 94%, 95%, 96%, 97%, 98%, 99%, or 100% sequence identity to 8H DNA (SEQ ID NO: 157). In certain embodiments, a polynucleotide of the present disclosure comprises a portion having at least 80%, 85%, 90%, 91%, 92%, 93%, 94%, 95%, 96%, 97%, 98%, 99%, or 100% sequence identity to 9H DNA (SEQ ID NO: 158). In certain embodiments, a polynucleotide of the present disclosure comprises a portion having at least 80%, 85%, 90%, 91%, 92%, 93%, 94%, 95%, 96%, 97%, 98%, 99%, or 100% sequence identity to 13H DNA (SEQ ID NO: 159). In certain embodiments, a polynucleotide of the present disclosure comprises a portion having at least 80%, 85%, 90%, 91%, 92%, 93%, 94%, 95%, 96%, 97%, 98%, 99%, or 100% sequence identity to 19H DNA (SEQ ID NO: 160). In certain embodiments, a polynucleotide of the present disclosure comprises a portion having at least 80%, 85%, 90%, 91%, 92%, 93%, 94%, 95%, 96%, 97%, 98%, 99%, or 100% sequence identity to 29H DNA (SEQ ID NO: 161). In certain embodiments, a polynucleotide of the present disclosure comprises a portion having at least 80%, 85%, 90%, 91%, 92%, 93%, 94%, 95%, 96%, 97%, 98%, 99%, or 100% sequence identity to 37H DNA (SEQ ID NO: 162). In certain embodiments, a polynucleotide of the present disclosure comprises a portion having at least 80%, 85%, 90%, 91%, 92%, 93%, 94%, 95%, 96%, 97%, 98%, 99%, or 100% sequence identity to 40H DNA (SEQ ID NO: 163). In certain embodiments, a polynucleotide of the present disclosure comprises a portion having at least 80%, 85%, 90%, 91%, 92%, 93%, 94%, 95%, 96%, 97%, 98%, 99%, or 100% sequence identity to 55H DNA (SEQ ID NO: 164). In certain embodiments, a polynucleotide of the present disclosure comprises a portion having at least 80%, 85%, 90%, 91%, 92%, 93%, 94%, 95%, 96%, 97%, 98%, 99%, or 100% sequence identity to 60H DNA (SEQ ID NO: 165). In certain embodiments, a polynucleotide of the present disclosure comprises a portion having at least 80%, 85%, 90%, 91%, 92%, 93%, 94%, 95%, 96%, 97%, 98%, 99%, or 100% sequence identity to 80H DNA (SEQ ID NO: 166). In certain embodiments, a polynucleotide of the present disclosure comprises a portion having at least 80%, 85%, 90%, 91%, 92%, 93%, 94%, 95%, 96%, 97%, 98%, 99%, or 100% sequence identity to 93H DNA (SEQ ID NO: 167). In certain embodiments, a polynucleotide of the present disclosure comprises a portion having at least 80%, 85%, 90%, 91%, 92%, 93%, 94%, 95%, 96%, 97%, 98%, 99%, or 100% sequence identity to 110H DNA (SEQ ID NO: 168). In certain embodiments, a polynucleotide of the present disclosure comprises a portion having at least 80%, 85%, 90%, 91%, 92%, 93%, 94%, 95%, 96%, 97%, 98%, 99%, or 100% sequence identity to 128H DNA (SEQ ID NO: 169).

In certain embodiments, a polynucleotide of the present disclosure comprises a portion having at least 80%, 85%, 90%, 91%, 92%, 93%, 94%, 95%, 96%, 97%, 98%, 99%, or 100% sequence identity to 8L DNA (SEQ ID NO: 170). In certain embodiments, a polynucleotide of the present disclosure comprises a portion having at least 80%, 85%, 90%, 91%, 92%, 93%, 94%, 95%, 96%, 97%, 98%, 99%, or 100% sequence identity to 9L DNA (SEQ ID NO: 171). In certain embodiments, a polynucleotide of the present disclosure comprises a portion having at least 80%, 85%, 90%, 91%, 92%, 93%, 94%, 95%, 96%, 97%, 98%, 99%, or 100% sequence identity to 13L DNA (SEQ ID NO: 172). In certain embodiments, a polynucleotide of the present disclosure comprises a portion having at least 80%, 85%, 90%, 91%, 92%, 93%, 94%, 95%, 96%, 97%, 98%, 99%, or 100% sequence identity to 19L DNA (SEQ ID NO: 173). In certain embodiments, a polynucleotide of the present disclosure comprises a portion having at least 80%, 85%, 90%, 91%, 92%, 93%, 94%, 95%, 96%, 97%, 98%, 99%, or 100% sequence identity to 29L DNA (SEQ ID NO: 174). In certain embodiments, a polynucleotide of the present disclosure comprises a portion having at least 80%, 85%, 90%, 91%, 92%, 93%, 94%, 95%, 96%, 97%, 98%, 99%, or 100% sequence identity to 37L DNA (SEQ ID NO: 175). In certain embodiments, a polynucleotide of the present disclosure comprises a portion having at least 80%, 85%, 90%, 91%, 92%, 93%, 94%, 95%, 96%, 97%, 98%, 99%, or 100% sequence identity to 40L DNA (SEQ ID NO: 176). In certain embodiments, a polynucleotide of the present disclosure comprises a portion having at least 80%, 85%, 90%, 91%, 92%, 93%, 94%, 95%, 96%, 97%, 98%, 99%, or 100% sequence identity to 55L DNA (SEQ ID NO: 177). In certain embodiments, a polynucleotide of the present disclosure comprises a portion having at least 80%, 85%, 90%, 91%, 92%, 93%, 94%, 95%, 96%, 97%, 98%, 99%, or 100% sequence identity to 60L DNA (SEQ ID NO: 178). In certain embodiments, a polynucleotide of the present disclosure comprises a portion having at least 80%, 85%, 90%, 91%, 92%, 93%, 94%, 95%, 96%, 97%, 98%, 99%, or 100% sequence identity to 80L DNA (SEQ ID NO: 179). In certain embodiments, a polynucleotide of the present disclosure comprises a portion having at least 80%, 85%, 90%, 91%, 92%, 93%, 94%, 95%, 96%, 97%, 98%, 99%, or 100% sequence identity to 93L DNA (SEQ ID NO: 180). In certain embodiments, a polynucleotide of the present disclosure comprises a portion having at least 80%, 85%, 90%, 91%, 92%, 93%, 94%, 95%, 96%, 97%, 98%, 99%, or 100% sequence identity to 110L DNA (SEQ ID NO: 181). In certain embodiments, a polynucleotide of the present disclosure comprises a portion having at least 80%, 85%, 90%, 91%, 92%, 93%, 94%, 95%, 96%, 97%, 98%, 99%, or 100% sequence identity to 128H DNA (SEQ ID NO: 182).

### Treatment of Diseases

Certain aspects of the present embodiments can be used to prevent or treat a neurologic or neurodegenerative disease and disorder including, but, not limited to, Alzheimer's Disease (AD), Parkinson's Disease (PD, dementia, dementia with Lewy bodies (DLB) and others, including neuroinflammatory processes and those involving microglia or a disease or disorder (e.g., a disease or disorder of the brain) associated with LILRB2-regulated proteins. LILRB2 activity may be increased or reduced by any LILRB2-binding antibodies. Preferably, such antibodies would be an anti-LILRB2 antibody.

“Treatment” and “treating” refer to administration or application of a therapeutic agent to a subject or performance of a procedure or modality on a subject for the purpose of obtaining a therapeutic benefit of a disease or health-related condition. For example, a treatment may include administration of a pharmaceutically effective amount of an antibody that modulates LILRB2 biological activity.

Treatment may be accomplished by perfusion, direct subcutaneous injection or injection into the blood circulation system.

“Subject” and “patient” refer to either a human or non-human, such as primates, mammals, and vertebrates. In particular embodiments, the subject is a human.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease.

### Chimeric Antigen Receptors

As used herein the term “chimeric antigen receptor” or “CAR” refers to an artificially constructed hybrid protein or polypeptide containing an antigen-binding domain of an antibody (e.g., a single chain variable fragment (scFv)) linked to a domain or signaling, e.g., T-cell signaling or T-cell activation domains, that activates an immune cell, e.g., a T cell or a NK cell. CARs are capable of redirecting the immune cell specificity and reactivity toward a selected target in a non-MHC-restricted manner, taking advantage of the antigen-binding properties of monoclonal antibodies. This non-MHC-restricted antigen recognition confers on immune cells expressing CARs the ability to recognize an antigen independent of processing, thus bypassing a mechanism of tumor escape. In another aspect, provided is a chimeric antigen receptor (CAR) protein comprising an antigen-binding fragment as provided herein. In another aspect, provided is an isolated nucleic acid that encodes a CAR protein as provided herein.

In another aspect, an engineered cell comprising the isolated nucleic acid as provided herein. In certain embodiments, the engineered cell is a T cell, NK cell, or myeloid cell. In another aspect, the present disclosure provides immune cells which express a chimeric antigen receptor (CAR). In some embodiments, the CAR comprises an antigen-binding fragment provided herein. In some embodiments, the CAR protein includes from the N-terminus to the C-terminus: a leader peptide, an anti-LILRB2 heavy chain variable domain, a linker domain, an anti-LILRB2 light chain variable domain, a human IgG1-CH2-CH3 domain, a spacer region, a CD28 transmembrane domain, an anti-LILRB2 intracellular co-stimulatory signaling and a CD35 intracellular T cell signaling domain.

In certain embodiments, the chimeric antigen receptor comprising an antigen-binding domain at least 80%, 85%, 90%, 91%, 92%, 93%, 94%, 95%, 96%, 97%, 98%, 99% or 100% identical to the antigen-binding domain of any one of the LILRB2-specific monoclonal antibodies disclosed herein. In certain embodiments, the engineered cell expresses an antigen-binding domain at least 80%, 85%, 90%, 91%, 92%, 93%, 94%, 95%, 96%, 97%, 98%, 99% or 100% identical to the antigen-binding domain of any one of the LILRB2-specific monoclonal antibodies disclosed herein.

In some embodiments, provided is a method of treating or ameliorating the effect of neurodegenerative diseases and disorders including, but, not limited to, dementia, Alzheimer's Disease (AD), Parkinson's Disease (PD), dementia with Lewy bodies (DLB) and others, including neuroinflammatory processes and those involving microglia in a subject, the method comprising administering to the subject a therapeutically effective amount of the antibody or an antigen-binding fragment thereof as defined herein.

In some embodiments, provided is a method of treating or ameliorating the effect of Alzheimer's Disease (AD) in a subject, the method comprising administering to the subject a therapeutically effective amount of the antibody or an antigen-binding fragment thereof as defined herein.

### Pharmaceutical Preparations

Where clinical application of a therapeutic composition containing an inhibitory antibody is undertaken, it will generally be beneficial to prepare a pharmaceutical or therapeutic composition appropriate for the intended application. In certain embodiments, pharmaceutical compositions may comprise, for example, at least about 0.1% of an active compound. In other embodiments, an active compound may comprise between about 2% to about 75% of the weight of the unit, or between about 25% to about 60%, for example, and any range derivable therein.

The therapeutic compositions of the present embodiments are advantageously administered in the form of injectable compositions either as liquid solutions or suspensions; solid forms suitable for solution in, or suspension in, liquid prior to injection may also be prepared. These preparations also may be emulsified.

The phrases “pharmaceutical or pharmacologically acceptable” refers to molecular entities and compositions that do not produce an adverse, allergic, or other untoward reaction when administered to an animal, such as a human, as appropriate. The preparation of a pharmaceutical composition comprising an antibody or additional active ingredient will be known to those of skill in the art in light of the present disclosure. Moreover, for animal (e.g., human) administration, it will be understood that preparations should meet sterility, pyrogenicity, general safety, and purity standards as required by FDA Office of Biological Standards.

As used herein, “pharmaceutically acceptable carrier” includes any and all aqueous solvents (e.g., water, alcoholic/aqueous solutions, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. The pH and exact concentration of the various components in a pharmaceutical composition are adjusted according to well-known parameters.

The active compounds can be formulated for parenteral administration, e.g., formulated for injection via the intravenous, intramuscular, sub-cutaneous, or even intraperitoneal routes. Typically, such compositions can be prepared as either liquid solutions or suspensions; solid forms suitable for use to prepare solutions or suspensions upon the addition of a liquid prior to injection can also be prepared; and the preparations can also be emulsified.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The proteinaceous compositions may be formulated into a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

A pharmaceutical composition can include a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

### Kits and Diagnostics

In various aspects of the embodiments, a kit is envisioned containing therapeutic agents and/or other therapeutic and delivery agents. In some embodiments, the present embodiments contemplate a kit for preparing and/or administering a therapy of the embodiments. The kit may comprise one or more sealed vials containing any of the pharmaceutical compositions of the present embodiments. The kit may include, for example, at least one anti-LILRB2 antibody as well as reagents to prepare, formulate, and/or administer the components of the embodiments or perform one or more steps of the inventive methods. In some embodiments, the kit may also comprise a suitable container, which is a container that will not react with components of the kit, such as an Eppendorf tube, an assay plate, a syringe, a bottle, or a tube. The container may be made from sterilizable materials such as plastic or glass.

The kit may further include an instruction sheet that outlines the procedural steps of the methods set forth herein, and will follow substantially the same procedures as described herein or are known to those of ordinary skill in the art. The instruction information may be in a computer readable media containing machine-readable instructions that, when executed using a computer, cause the display of a real or virtual procedure of delivering a pharmaceutically effective amount of a therapeutic agent.

It is understood that wherever embodiments are described herein with the language “comprising,” otherwise analogous embodiments described in terms of “consisting of” and/or “consisting essentially of” are also provided.

Where aspects or embodiments of the invention are described in terms of a Markush group or other grouping of alternatives, the present invention encompasses not only the entire group listed as a whole, but each member of the group individually and all possible subgroups of the main group, but also the main group absent one or more of the group members. The present invention also envisages the explicit exclusion of one or more of any of the group members in the claimed invention.

Unless otherwise defined, all technical and scientific terms used herein have the same meaning as commonly understood by one of ordinary skill in the art to which this invention belongs. In case of conflict, the present specification, including definitions, will control. Throughout this specification and claims, the word “comprise,” or variations such as “comprises” or “comprising” will be understood to imply the inclusion of a stated integer or group of integers but not the exclusion of any other integer or group of integers. Unless otherwise required by context, singular terms shall include pluralities and plural terms shall include the singular. Any example(s) following the term “e.g.” or “for example” is not meant to be exhaustive or limiting.

The practice of the present invention will employ, unless otherwise indicated, conventional techniques of molecular biology (including recombinant techniques), microbiology, cell biology, biochemistry and immunology, which are within the skill of the art.

The present invention is illustrated by the following examples. It is to be understood that the particular examples, materials, amounts, and procedures are to be interpreted broadly in accordance with the scope and spirit of the invention as set forth herein.

### EXAMPLES

The following examples are included to demonstrate certain embodiments of the invention.

Unless noted otherwise, data generated from the experiments and Examples described hereinbelow can be found in Zhao et al. Molecular Neurodegeneration (2022) 17:44; which is incorporated herein by reference in its entirety.)

### Experimental Procedures and Methods

### Example 1. Production of Polynucleotides Encoding LILRB2-Binding Monoclonal Antibodies

Human phage antibody libraries were panned with recombinantly-expressed LILRB2-His select, enrich and isolate high-affinity LILRB2-binding bacteriophage. The DNA sequence of each bacteriophage clone was determined and the sequences were analyzed using GeneBank IgBLAST to identify germline V(D)J gene segments. Individual V_{H} and V_{L} genes were mapped to the germline of major IGL and IGH locus. CDR sequences were annotated according to IMGT (http://www.imgt.org/) nomenclature.

DNA fragments encoding V_{H} and V_{L} chains were amplified by PCR using gene specific primers. The PCR products of V_{H} and V_{L} gene fragments were gel-extracted and purified to make full length heavy chain (HC) and light chain (LC) DNA constructs using an infusion cloning kit (In-Fusion® HD Cloning kit, Clontech).

Table 1 shows the binding of particular clones from the phage display library to LILRB2 in an ELISA assay.

TABLE 1

| mAb | OD₄₅₀ in |
|---|---|
| Designation | ELISA assay |
| NeuB2-8 | 0.5481 |
| NeuB2-9 | 0.5152 |
| NeuB2-13 | 0.5517 |
| NeuB2-19 | 0.6628 |
| NeuB2-29 | 0.8188 |
| NeuB2-37 | 0.4166 |
| NeuB2-40 | 0.2756 |
| NeuB2-55 | 0.8861 |
| NeuB2-60 | 0.7442 |
| NeuB2-80 | 0.6397 |
| NeuB2-93 | 0.5426 |
| NeuB2-110 | 0.9722 |
| NeuB2-128 | 0.4459 |

### Example 2. Expression of Anti-LILRB2 Monoclonal Antibodies

Human anti-LILRB2 antibodies were produced in mammalian cells (Expi 293 cells from Thermo Fisher) by transiently transfecting HEK293 cells with paired HC and LC containing DNA constructs. Antibodies in the culture medium were purified (isolated) using Protein A resin according to a method based on the manufacturer's (Repligen) instructions.

### mAb Characterization—Affinity Measurements and Kinetic Binding Property Using Bio-Layer Interferometry (BLI)

Kinetic binding parameters (KD, kₒₙ, and k_{off}) were determined using Octet Red96 instrument (ForteBio; Fremont, CA). The results are shown in Table 2.

TABLE 2
Kinetic binding parameters of LILRB2 mAbs determined using
biolayer interferometry (BLI) assay with Octet Red96.

| mAb | | K_{D} | kₒₙ | k_{dis} | Full |
|---|---|---|---|---|---|
| Name | Label | (M) | (1/Ms) | (1/s) | R² |
| NeuB2-29 | Ab29 | 9.00E−09 | 1.68E+05 | 1.51E−03 | 0.9879 |
| NeuB2-37 | Ab37 | 8.45E−09 | 4.74E+04 | 4.00E−04 | 0.9967 |
| NeuB2-40 | Ab40 | 1.92E−08 | 2.45E+05 | 4.71E−03 | 0.9681 |
| NeuB2-55 | Ab55 | 5.60E−08 | 1.29E+05 | 7.23E−03 | 0.9939 |
| NeuB2-60 | Ab60 | 2.41E−08 | 2.12E+05 | 5.12E−03 | 0.9856 |
| NeuB2-93 | Ab93 | 8.39E−08 | 1.23E+05 | 1.03E−02 | 0.9952 |

### mAb Characterization—Screening Antibody Blocking Ligand Activation in a LILRB2 Reporter Cell System

Plate-coated oAβ was incubated with LILRB2-chimeric reporter cells in the presence of increasing concentrations of purified NeuB2-mAbs and GFP signals in the reporter cells were determined using flow cytometry (iQue3 instrument). IC₅₀ values were calculated using non-linear curve fitting function log (inhibitor) vs. response in GraphPad Prism 8.0. The results are shown in Tables 3 and 4.

TABLE 3
Titration of blocking activities of purified LILRB2
antibodies against oAβ LILRB2 interactions.

| mAb Name | IC₅₀ (M) | IC₅₀ 95% CI (M) | R² |
|---|---|---|---|
| NeuB2-29 | 3.44E−10 | 2.277e−010 to | 0.9994 |
| | | 5.181e−010 | |
| NeuB2-37 | 8.62E−10 | 8.212e−010 to | 0.9980 |
| | | 9.020e−010 | |
| NeuB2-40 | 3.6E−09 | 3.385e−009 to | 0.9979 |
| | | 3.838e−009 | |
| NeuB2-55 | 1.89E−09 | 1.588e−009 to | 0.9896 |
| | | 2.271e−009 | |
| NeuB2-60 | 9.82E−10 | 8.943e−010 to | 0.9950 |
| | | 1.079e−009 | |
| NeuB2-93 | 3.81E−09 | 3.506e−009 to | 0.9972 |
| | | 4.154e−009 | |

TABLE 4
Titration of blocking activities of purified LILRB2
antibodies against PS-LILRB2 interactions.

| mAb Name | IC₅₀ (M) | IC₅₀ 95% CI (M) | R² |
|---|---|---|---|
| NeuB2-29 | 1.415E−10 | 1.345e−010 to | 0.9982 |
| | | 1.490e−010 | |
| NeuB2-37 | 2.929E−10 | 2.663e−010 to | 0.9919 |
| | | 3.190e−010 | |
| NeuB2-40 | 3.535E−10 | 3.138e−010 to | 0.9942 |
| | | 3.993e−010 | |
| NeuB2-55 | 3.35E−10 | 3.004e−010 to | 0.9930 |
| | | 3.765e−010 | |
| NeuB2-60 | 3.214E−10 | 3.004e−010 to | 0.9957 |
| | | 3.449e−010 | |
| NeuB2-93 | 4.448E−10 | 3.864e−010 to | 0.9937 |
| | | 5.119e−010 | |

### mAb Characterization—Affinity Measurement and EC₅₀ Determination Using ELISA.

Antibody binding affinity to LILRB2 were measured by ELISA. Increasing concentrations of purified LILRB2 antibodies were incubated with plate-coated LILRB2. The bound antibody was detected by anti-human F(ab)2 HRP, and the signals were quantified as OD₄₅₀ values. EC₅₀ values (Table 5) were calculated using non-linear curve fitting function Sigmoidal dose-response (variable slope) in GraphPad Prism.

TABLE 5
Affinity measurement and EC₅₀ determination.

| mAb Name | EC₅₀ (M) | EC₅₀ 95% CI (M) | R² |
|---|---|---|---|
| NeuB2-29 | 1.374E−10 | 1.301e−010 to 1.453e−010 | 0.9990 |
| NeuB2-37 | 6.585E−10 | 5.971e−010 to 7.365e−010 | 0.9991 |
| NeuB2-40 | 6.645E−11 | 6.222e−011 to 7.096e−011 | 0.9987 |
| NeuB2-55 | 1.753E−10 | 1.643e−010 to 1.873e−010 | 0.9988 |
| NeuB2-60 | 1.61E−10 | 1.493e−010 to 1.738e−010 | 0.9983 |
| NeuB2-93 | 1.589E−10 | 1.529e−010 to 1.652e−010 | 0.9996 |

### SEQUENCE LISTING

TABLE 6
Amino acid sequences of Heavy Chain CDRs

| | | |
|---|---|---|
| 8HC-CDR1-AA | (SEQ ID NO: 1) | GGNVSSKSAA |
| (NeuB2-8) | | |
| 8HC-CDR2-AA | (SEQ ID NO: 2) | TYYRSKWYN |
| (NeuB2-8) | | |
| 8HC-CDR3-AA | (SEQ ID NO: 3) | ARSLGEGWFPYFYGMDV |
| (NeuB2-8) | | |
| 9HC-CDR1-AA | (SEQ ID NO: 4) | GYTFTSYA |
| (NeuB2-9) | | |
| 9HC-CDR2-AA | (SEQ ID NO: 5) | INAGNGNT |
| (NeuB2-9) | | |
| 9HC-CDR3-AA | (SEQ ID NO: 6) | ARDFMGYCSSTSCYGVTGAFDI |
| (NeuB2-9) | | |
| 13HC-CDR1-AA | (SEQ ID NO: 7) | GYTFTSYY |
| (NeuB2-13) | | |
| 13HC-CDR2-AA | (SEQ ID NO: 8) | INPSGGST |
| (NeuB2-13) | | |
| 13HC-CDR3-AA | (SEQ ID NO: 9) | AREDWGPFDY |
| (NeuB2-13) | | |
| 19HC-CDR1-AA | (SEQ ID NO: 10) | GFTFDDYG |
| (NeuB2-19) | | |
| 19HC-CDR2-AA | (SEQ ID NO: 11) | INWNGGST |
| (NeuB2-19) | | |
| 19HC-CDR3-AA | (SEQ ID NO: 12) | AREGDGAFDI |
| (NeuB2-19) | | |
| 29HC-CDR1-AA | (SEQ ID NO: 13) | GFTFSDYA |
| (NeuB2-29) | | |
| 29HC-CDR2-AA | (SEQ ID NO: 14) | ISYDGSQK |
| (NeuB2-29) | | |
| 29HC-CDR3-AA | (SEQ ID NO: 15) | AKFPSLNDNVGFDV |
| (NeuB2-29) | | |
| 37HC-CDR1-AA | (SEQ ID NO: 16) | GGTFSSYA |
| (NeuB2-37) | | |
| 37HC-CDR2-AA | (SEQ ID NO: 17) | IIPIFGTA |
| (NeuB2-37) | | |
| 37HC-CDR3-AA | (SEQ ID NO: 18) | ARGNHVLRFLEWLPNWFDP |
| (NeuB2-37) | | |
| 40HC-CDR1-AA | (SEQ ID NO: 19) | GYTFTNYG |
| (NeuB2-40) | | |
| 40HC-CDR2-AA | (SEQ ID NO: 20) | ISGDAGDT |
| (NeuB2-40) | | |
| 40HC-CDR3-AA | (SEQ ID NO: 21) | ARDGEYIPMFRGFDNYYGLDV |
| (NeuB2-40) | | |
| 55HC-CDR1-AA | (SEQ ID NO: 22) | GGTFSSYA |
| (NeuB2-55) | | |
| 55HC-CDR2-AA | (SEQ ID NO: 23) | IIPIFGTA |
| (NeuB2-55) | | |
| 55HC-CDR3-AA | (SEQ ID NO: 24) | ARGVDQVDY |
| (NeuB2-55) | | |
| 60HC-CDR1-AA | (SEQ ID NO: 25) | GGSITSSNW |
| (NeuB2-60) | | |
| 60HC-CDR2-AA | (SEQ ID NO: 26) | ISHSGST |
| (NeuB2-60) | | |
| 60HC-CDR3-AA | (SEQ ID NO: 27) | ARAFHGMDV |
| (NeuB2-60) | | |
| 80HC-CDR1-AA | (SEQ ID NO: 28) | GYTFTSYG |
| (NeuB2-80) | | |
| 80HC-CDR2-AA | (SEQ ID NO: 29) | ISAYNGNT |
| (NeuB2-80) | | |
| 80HC-CDR3-AA | (SEQ ID NO: 30) | ARDGGGGYFDY |
| (NeuB2-80) | | |
| 93HC-CDR1-AA | (SEQ ID NO: 31) | GGRFSSNV |
| (NeuB2-93) | | |
| 93HC-CDR2-AA | (SEQ ID NO: 32) | IIPIFGTP |
| (NeuB2-93) | | |
| 93HC-CDR3-AA | (SEQ ID NO: 33) | ASGSTPFDAFDI |
| (NeuB2-93) | | |
| 110HC-CDR1- | (SEQ ID NO: 34) | GFTFSDYY |
| AA | | |
| (NeuB2-110) | | |
| 110HC-CDR2- | (SEQ ID NO: 35) | IGTSGSTI |
| AA | | |
| (NeuB2-110) | | |
| 110HC-CDR3- | (SEQ ID NO: 36) | AREATSGTYFDY |
| AA | | |
| (NeuB2-110) | | |
| 128HC-CDR1- | (SEQ ID NO: 37) | GGTFSSYA |
| AA | | |
| (NeuB2-128) | | |
| 128HC-CDR2- | (SEQ ID NO: 38) | IIPIFGTA |
| AA | | |
| (NeuB2-128) | | |
| 128HC-CDR3- | (SEQ ID NO: 39) | ARGPSGSPYGMDV |
| AA | | |
| (NeuB2-128) | | |

TABLE 7
Amino Acid Sequences of Light Chain CDRs

| Sequence Label | | |
|---|---|---|
| (mAb Source) | Sequence ID | Amino Acid Sequence |
| 8LC-CDR1-AA | (SEQ ID NO: 40) | NRDVGGYDY |
| (NeuB2-8) | | |
| 8LC-CDR3-AA | (SEQ ID NO: 41) | SSYTASRTVV |
| (NeuB2-8) | | |
| 9LC-CDR1-AA | (SEQ ID NO: 42) | SSSIGSNP |
| (NeuB2-9) | | |
| 9LC-CDR3-AA | (SEQ ID NO: 43) | AAWDDSLNGDYV |
| (NeuB2-9) | | |
| 13LC-CDR1-AA | (SEQ ID NO: 44) | QSISNF |
| (NeuB2-13) | | |
| 13LC-CDR3-AA | (SEQ ID NO: 45) | QQGYSVPLT |
| (NeuB2-13) | | |
| 19LC-CDR1-AA | (SEQ ID NO: 46) | GSNIRSNG |
| (NeuB2-19) | | |
| 19LC-CDR3-AA | (SEQ ID NO: 47) | AAWDDNLNAWV |
| (NeuB2-19) | | |
| 29LC-CDR1-AA | (SEQ ID NO: 48) | TSNIENNA |
| (NeuB2-29) | | |
| 29LC-CDR3-AA | (SEQ ID NO: 49) | AVWDDSLNGPV |
| (NeuB2-29) | | |
| 37LC-CDR1-AA | (SEQ ID NO: 50) | SSNIGSNT |
| (NeuB2-237) | | |
| 37LC-CDR3-AA | (SEQ ID NO: 51) | AAWDDSLNGHV |
| (NeuB2-37) | | |
| 40LC-CDR1-AA | (SEQ ID NO: 52) | SSNIGKNA |
| (NeuB2-40) | | |
| 40LC-CDR3-AA | (SEQ ID NO: 53) | AAWDDRLKGYV |
| (NeuB2-40) | | |
| 55LC-CDR1-AA | (SEQ ID NO: 54) | SSDVGGYNY |
| (NeuB2-55) | | |
| 55LC-CDR3-AA | (SEQ ID NO: 55) | SSYTSSSTPYV |
| (NeuB2-55) | | |
| 60LC-CDR1-AA | (SEQ ID NO: 56) | SSNIANNA |
| (NeuB2-60) | | |
| 60LC-CDR3-AA | (SEQ ID NO: 57) | ATWDDDLNGPI |
| (NeuB2-60) | | |
| 80LC-CDR1-AA | (SEQ ID NO: 58) | SSNIGKNI |
| (NeuB2-80) | | |
| 80LC-CDR3-AA | (SEQ ID NO: 59) | AAWDDSLRSWL |
| (NeuB2-80) | | |
| 93LC-CDR1-AA | (SEQ ID NO: 60) | SSDVGGYNY |
| (NeuB2-93) | | |
| 93LC-CDR3-AA | (SEQ ID NO: 61) | SSYTSSSTPWV |
| (NeuB2-93) | | |
| 110LC-CDR1- | (SEQ ID NO: 62) | SSDVGGYNY |
| AA | | |
| (NeuB2-110) | | |
| 110LC-CDR3- | (SEQ ID NO: 63) | SSYTSSSTPYV |
| AA | | |
| (NeuB2-110) | | |
| 128LC-CDR1- | (SEQ ID NO: 64) | QDISNY |
| AA | | |
| (NeuB2-128) | | |
| 128LC-CDR3- | (SEQ ID NO: 65) | QQYDNLPLT |
| AA | | |
| (NeuB2-128) | | |

TABLE 8
Amino Acid sequences of antibody heavy (H) chains

| Sequence | | |
|---|---|---|
| Label | | |
| (mAb | Sequence | |
| Source) | ID | Amino Acid Sequence |
| 8H-AA | (SEQ ID | QLQLQESGPGLVKPSQTLSLTCAISGGNVSSKSAAWN |
| (NeuB2-8) | NO: 66) | WIRQSPSRGLEWLGRTYYRSKWYNDYAVSVKSRITIN |
| | | PDTSKNQFSLQLKSVTPEDTAVYYCARSLGEGWFPYF |
| | | YGMDVWGQGTTVTVSS |
| 9H-AA | (SEQ ID | QVQLVQSGAEVKKPGASVKVSCKASGYTFTSYAMH |
| (NeuB2-9) | NO: 67) | WVRQAPGQRLEWMGWINAGNGNTKYSQKFQGRVTI |
| | | TRDTSASTAYMELSSLRSEDTAVYYCARDFMGYCSST |
| | | SCYGVTGAFDIWGQGTMVTVSS |
| 13H-AA | (SEQ ID | QVQLVQSGAEVKKPGASVKVSCKASGYTFTSYYMH |
| (NeuB2- | NO: 68) | WVRQAPGQGLEWMGIINPSGGSTSYAQKFQGRVTMT |
| 13) | | RDTSTSTVYMELSSLRSEDTAVYYCAREDWGPFDYW |
| | | GQGTTVTVSS |
| 19H-AA | (SEQ ID | QVQLVESGGGVVRPGGSLRLSCAASGFTFDDYGMSW |
| (NeuB2- | NO: 69) | VRQAPGKGLEWVSGINWNGGSTGYADSVKGRFTISR |
| 19) | | DNAKNSLYLQMNSLRAEDTALYYCAREGDGAFDIWG |
| | | QGTLVTVSS |
| 29H-AA | (SEQ ID | QVQLVETGGGVVQPGRSLRLSCAASGFTFSDYAMHW |
| (NeuB2- | | VRQAPGKGLEWVTLISYDGSQKYYADSVKGRFTISRD |
| 29) | NO: 70) | DSKNTVDLQMNSLKAEDTAVYYCAKFPSLNDNVGFD |
| | | VWGQGTMVTVSS |
| 37H-AA | (SEQ ID | QVQLMQSGAEVKKPGSSVKVSCKASGGTFSSYAISWV |
| (NeuB2- | NO: 71) | RQAPGQGLEWMGGIIPIFGTANYAQKFQGRVTITADES |
| 37) | | TSTAYMELSSLRSEDTAVYYCARGNHVLRFLEWLPN |
| | | WFDPWGQGTLVTVSS |
| 40H-AA | (SEQ ID | QVQLVQSGAEVKKPGASVKVSCKASGYTFTNYGISW |
| (NeuB2- | NO: 72) | VRQAPGQGLEWMGWISGDAGDTKFAQKFQGRVTMT |
| 40) | | TDTSTTTTYMELRSLRSDDTAVYYCARDGEYIPMFRG |
| | | FDNYYGLDVWGQGTTVTVSS |
| 55H-AA | (SEQ ID | EVQLVQSGAEVKKPGSSVKVSCKASGGTFSSYAISWV |
| (NeuB2- | NO: 73) | RQAPGQGLEWMGGIIPIFGTANYAQKFQGRVTITADES |
| 55) | | TSTAYMELSSLRSEDTAVYYCARGVDQVDYWGQGTL |
| | | VTVSS |
| 60H-AA | (SEQ ID | QVQLQESGPGLVKPSGTLSLTCAVSGGSITSSNWWSW |
| (NeuB2- | NO: 74) | VRQPPGKGLEWIGDISHSGSTNYNPSLKGRVTMSVDR |
| 60) | | SKNQFFLKLSSVTAADTAMYYCARAFHGMDVWGQG |
| | | TTVTVSS |

TABLE 9
Amino Acid sequences of antibody light (L) chains

| Sequence | | |
|---|---|---|
| Label | Sequence | |
| (mAb Source) | ID | Amino Acid Sequence |
| 8H-AA | (SEQ ID | QSVLTQPASVSGSPGQSITISCTGTNRDVGGYDY |
| (NeuB2-8) | NO: 79) | VSWYQQHPGKAPKLMIYGVSNRPSGISNRFSGSK |
| | | SGSTASLTISGLQVEDEAAYYCSSYTASRTVVFG |
| | | GGTKVTVL |
| 9H-AA | (SEQ ID | QSVLTQPPSASGTPGQRVTISCSGSSSSIGSNPVN |
| (NeuB2-9) | NO: 80) | WYQQLPGTAPKLLIYSNNQRPSGVPDRFSGSKSG |
| | | TSPSLAISGLQSEDEADYYCAAWDDSLNGDYVF |
| | | GTGTKVTVL |
| 13H-AA | (SEQ ID | DIQMTQSPSSLSASVGDRVTITCRASQSISNFVNW |
| (NeuB2-13) | NO: 81) | YQQKPGEVPKLLIYGASSLQSGVPSRFSGSGSGT |
| | | DFTLTISSLQPEDFATYYCQQGYSVPLTFGGGTK |
| | | VEIK |
| 19H-AA | (SEQ ID | QAVVTQPPSASGTPGQRVTISCSGSGSNIRSNGVT |
| (NeuB2-19) | NO: 82) | WYQQFPGTAPKLLIYDNNQRPSGVPGRFSGSKSG |
| | | TSGSLAISGLQSEDEADYYCAAWDDNLNAWVFG |
| | | AGTQLTVL |
| 29H-AA | (SEQ ID | QSVLTQPPSVSGAPRERVTISCSGSTSNIENNAVN |
| (NeuB2-29) | NO: 83) | WYQQLPGKAPKLLIYYDDLVPSGVSDRFSGSKSG |
| | | TSASLAISGLQSGDEAYYYCAVWDDSLNGPVFG |
| | | GGTKVTVL |
| 37H-AA | (SEQ ID | QSVLTQPPSASGTPGQRVTISCSGSSSNIGSNTVN |
| (NeuB2-37) | NO: 84) | WYQQFPGTAPKLLIYSNNQRPSGVPDRFSGSKSG |
| | | TSASLAISGLQSEDEADYYCAAWDDSLNGHVFG |
| | | TGTKVTVL |
| 40H-AA | (SEQ ID | QPVLTQPPSVSEAPRQRVTISCSGSSSNIGKNAVN |
| (NeuB2-40) | NO: 85) | WYQQFPGKAPKLLIYYDDLLPSGVSDRFSGSRSG |
| | | TSASLAISGLQSEDEADYYCAAWDDRLKGYVFG |
| | | TGTQLTVL |
| 55H-AA | (SEQ ID | QAVVTQPASVSGSPGQSITISCTGTSSDVGGYNY |
| (NeuB2-55) | NO: 86) | VSWYQQHPGKAPKLMIYDVSNRPSGVSNRFSGS |
| | | KSGNTASLTISGLQAEDEADYYCSSYTSSSTPYVF |
| | | GTGTKVTVL |
| 60H-AA | (SEQ ID | QSVLTQPPSVSEAPRQRVTISCSGSSSNIANNAVN |
| (NeuB2-60) | NO: 87) | WYQQLPGKAPKLLIYYDDLLASGVSDRFSGSRSG |
| | | TSAALAISGLQSEDEADYYCATWDDDLNGPIFGG |
| | | GTKVTVL |
| 80H-AA | (SEQ ID | QAVVTQPPSASGTPGQRITISCSGSSSNIGKNIVN |
| (NeuB2-80) | NO: 88) | WYQQVPGAAPKLLIYNNSQRPSGVPDRFSGSKSG |
| | | TSASLAISGLQSEDEADYYCAAWDDSLRSWLFG |
| | | GGTKVTVL |
| 93H-AA | (SEQ ID | QPVLTQPASVSGSPGQSITISCTGTSSDVGGYNYV |
| (NeuB2-93) | NO: 89) | SWYQQHPGKAPKLMIYEVSNRPSGVSNRFSGSKS |
| | | GNTASLTISGLQAEDEADYYCSSYTSSSTPWVFG |
| | | GGTKVTVL |
| 110H-AA | (SEQ ID | QAVVTQPASVSGSPGQSITISCTGTSSDVGGYNY |
| (NeuB2-110) | NO: 90) | VSWYQQHPGKAPKLMIYEVTNRPSGVSNRFSGS |
| | | KSGNTASLTISGLQAEDEADYYCSSYTSSSTPYVF |
| | | GTGTKVTVL |
| 128H-AA | (SEQ ID | DIQMTQSPSSLSASVGDRVTITCQASQDISNYLN |
| (NeuB2-128) | NO: 91) | WYQQKPGKAPKLLIYDASNLETGVPSRFSGSGSG |
| | | TDFTFTISSLQPEDIATYYCQQYDNLPLTFGGGTK |
| | | VDIK |

TABLE 10
DNA sequences of Heavy Chain CDRs

| Sequence Label | Sequence ID | DNA Sequence |
|---|---|---|
| 8HC-CDR1-DNA | (SEQ ID NO: 92) | GGGGGCAATGTCTCTAGTAAAAGTG |
| | | CTGCT |
| 8HC-CDR2-DNA | (SEQ ID NO: 93) | ACATACTACAGGTCCAAGTGGTATA |
| | | AC |
| 8HC-CDR3-DNA | (SEQ ID NO: 94) | GCAAGAAGCCTGGGTGAGGGCTGGT |
| | | TCCCATACTTCTACGGTATGGACGTC |
| 9HC-CDR1-DNA | (SEQ ID NO: 95) | GGATACACCTTCACTAGCTATGCT |
| 9HC-CDR2-DNA | (SEQ ID NO: 96) | ATCAACGCTGGCAATGGTAACACA |
| 9HC-CDR3-DNA | (SEQ ID NO: 97) | GCGAGAGATTTTATGGGATATTGTA |
| | | GTAGTACCAGCTGCTATGGGGTTAC |
| | | TGGTGCTTTTGATATC |
| 13HC-CDR1-DNA | (SEQ ID NO: 98) | GGATACACCTTCACCAGCTACTAT |
| 13HC-CDR2-DNA | (SEQ ID NO: 99) | ATCAACCCTAGTGGTGGTAGCACA |
| 13HC-CDR3-DNA | (SEQ ID NO: 100) | GCGAGAGAGGATTGGGGTCCCTTTG |
| | | ACTAC |
| 19HC-CDR1-DNA | (SEQ ID NO: 101) | GGATTCACCTTTGATGATTATGGC |
| 19HC-CDR2-DNA | (SEQ ID NO: 102) | ATTAATTGGAATGGTGGTAGCACA |
| 19HC-CDR3-DNA | (SEQ ID NO: 103) | GCGAGAGAGGGGGACGGTGCTTTTG |
| | | ATATC |
| 29HC-CDR1-DNA | (SEQ ID NO: 104) | GGATTCACCTTCAGTGACTATGCT |
| 29HC-CDR2-DNA | (SEQ ID NO: 105) | ATATCATATGATGGAAGTCAAAAA |
| 29HC-CDR3-DNA | (SEQ ID NO: 106) | GCGAAGTTTCCGTCCTTAAATGACA |
| | | ACGTGGGTTTTGATGTC |
| 37HC-CDR1-DNA | (SEQ ID NO: 107) | GGAGGCACCTTCAGCAGCTATGCT |
| 37HC-CDR2-DNA | (SEQ ID NO: 108) | ATCATCCCTATCTTTGGTACAGCA |
| 37HC-CDR3-DNA | (SEQ ID NO: 109) | GCGAGAGGTAACCACGTATTACGAT |
| | | TTTTGGAGTGGTTACCCAACTGGTTC |
| | | GACCCC |
| 40HC-CDR1-DNA | (SEQ ID NO: 110) | GGTTACACCTTTACCAACTATGGT |
| 40HC-CDR2-DNA | (SEQ ID NO: 111) | ATCAGCGGTGACGCTGGTGACACA |
| 40HC-CDR3-DNA | (SEQ ID NO: 112) | GCGAGAGATGGGGAATATATTCCTA |
| | | TGTTTCGGGGATTTGACAACTACTA |
| | | CGGTCTGGACGTC |
| 55HC-CDR1-DNA | (SEQ ID NO: 113) | GGAGGCACCTTCAGCAGCTATGCT |
| 55HC-CDR2-DNA | (SEQ ID NO: 114) | ATCATCCCTATCTTTGGTACAGCA |
| 55HC-CDR3-DNA | (SEQ ID NO: 115) | GCGAGAGGCGTGGACCAGGTTGACT |
| | | AC |
| 60HC-CDR1-DNA | (SEQ ID NO: 116) | GGTGGCTCCATCACCAGTAGTAACT |
| | | GG |
| 60HC-CDR2-DNA | (SEQ ID NO: 117) | ATCTCTCATAGTGGGAGCACC |
| 60HC-CDR3-DNA | (SEQ ID NO: 118) | GCGAGAGCCTTTCACGGTATGGACG |
| | | TC |
| 80HC-CDR1-DNA | (SEQ ID NO: 119) | GGTTACACCTTTACCAGCTATGGT |
| 80HC-CDR2-DNA | (SEQ ID NO: 120) | ATCAGCGCTTACAATGGTAACACA |
| 80HC-CDR3-DNA | (SEQ ID NO: 121) | GCGAGAGATGGGGGTGGCGGGTACT |
| | | TTGACTAC |
| 93HC-CDR1-DNA | (SEQ ID NO: 122) | GGAGGCAGGTTCAGCAGCAATGTT |
| 93HC-CDR2-DNA | (SEQ ID NO: 123) | ATCATCCCTATTTTTGGTACACCA |
| 93HC-CDR3-DNA | (SEQ ID NO: 124) | GCGAGCGGTAGTACGCCTTTTGATG |
| | | CTTTTGATATC |
| 110HC-CDR1-DNA | (SEQ ID NO: 125) | GGATTCACCTTCAGTGACTACTAT |
| 110HC-CDR2-DNA | (SEQ ID NO: 126) | ATTGGTACTAGTGGTAGTACCATA |
| 110HC-CDR3-DNA | (SEQ ID NO: 127) | GCGAGAGAGGCCACCTCTGGAACCT |
| | | ACTTTGACTAC |
| 128HC-CDR1-DNA | (SEQ ID NO: 128) | GGAGGCACCTTCAGCAGCTATGCT |
| 128HC-CDR2-DNA | (SEQ ID NO: 129) | ATCATCCCTATCTTTGGTACAGCA |
| 128HC-CDR3-DNA | (SEQ ID NO: 130) | GCGAGAGGTCCGAGTGGTAGCCCTT |
| | | ACGGTATGGACGTC |

TABLE 11
DNA Sequences of Light Chain CDRs

| Sequence Label | Sequence ID | DNA Sequence |
|---|---|---|
| 8LC-CDR1-DNA | (SEQ ID NO: | AACAGAGACGTTGGTGGTTATGACT |
| (NeuB2-8) | 131) | AT |
| 8LC-CDR2-DNA | | GGTGTCAGT |
| (NeuB2-8) | | |
| 8LC-CDR3-DNA | (SEQ ID NO: | TCTTCATACACAGCCAGCCGCACTG |
| (NeuB2-8) | 132) | TGGTG |
| 9LC-CDR1-DNA | (SEQ ID NO: | AGCTCCAGCATCGGAAGTAATCCT |
| (NeuB2-9) | 133) | |
| 9LC-CDR2-DNA | | AGTAATAAT |
| (NeuB2-9) | | |
| 9LC-CDR3-DNA | (SEQ ID NO: | GCAGCATGGGATGACAGCCTGAATG |
| (NeuB2-9) | 134) | GCGATTATGTC |
| 13LC-CDR1-DNA | (SEQ ID NO: 135) | CAGAGCATAAGCAACTTT |
| (NeuB2-13) | | |
| 13LC-CDR2-DNA | | GGTGCATCC |
| (NeuB2-13) | | |
| 13LC-CDR3-DNA | (SEQ ID NO: | CAACAGGGTTACAGTGTCCCACTCA |
| (NeuB2-13) | 136) | CT |
| 19LC-CDR1-DNA | (SEQ ID NO: | GGCTCCAACATCAGAAGTAATGGT |
| (NeuB2-19) | 137) | |
| 19LC-CDR2-DNA | | GATAATAAT |
| (NeuB2-19) | | |
| 19LC-CDR3-DNA | (SEQ ID NO: | GCAGCATGGGATGACAACCTGAATG |
| (NeuB2-19) | 138) | CTTGGGTG |
| 29LC-CDR1-DNA | (SEQ ID NO: | ACCTCCAATATCGAAAATAATGCT |
| (NeuB2-29) | 139) | |
| 29LC-CDR2-DNA | | TATGATGAT |
| (NeuB2-29) | | |
| 29LC-CDR3-DNA | (SEQ ID NO: | GCAGTTTGGGATGACAGCCTGAATG |
| (NeuB2-29) | 140) | GTCCGGTT |
| 37LC-CDR1-DNA | (SEQ ID NO: | AGCTCCAACATCGGAAGTAATACT |
| (NeuB2-237) | 141) | |
| 37LC-CDR2-DNA | | AGTAATAAT |
| (NeuB2-37) | | |
| 37LC-CDR3-DNA | (SEQ ID NO: | GCAGCATGGGATGACAGCCTGAATG |
| (NeuB2-37) | 142) | GTCATGTC |
| 40LC-CDR1-DNA | (SEQ ID NO: | AGCTCCAACATCGGAAAGAATGCT |
| (NeuB2-40) | 143) | |
| 40LC-CDR2-DNA | | TATGATGAT |
| (NeuB2-40) | | |
| 40LC-CDR3-DNA | (SEQ ID NO: | GCAGCATGGGATGACAGACTGAAA |
| (NeuB2-40) | 144) | GGATACGTC |
| 55LC-CDR1-DNA | (SEQ ID NO: | AGCAGTGACGTTGGTGGTTATAACT |
| (NeuB2-55) | 145) | AT |
| 55LC-CDR2-DNA | | GATGTCAGT |
| (NeuB2-55) | | |
| 55LC-CDR3-DNA | (SEQ ID NO: | AGCTCATATACAAGCAGCAGCACCC |
| (NeuB2-55) | 146) | CTTATGTC |
| 60LC-CDR1-DNA | (SEQ ID NO: | AGCTCCAATATCGCAAATAATGCT |
| (NeuB2-60) | 147) | |
| 60LC-CDR2-AA | | TATGATGAT |
| (NeuB2-60) | | |
| 60LC-CDR3-DNA | (SEQ ID NO: 148) | GCAACATGGGATGACGACCTGAATG |
| (NeuB2-60) | | GTCCGATA |
| 80LC-CDR1-DNA | (SEQ ID NO: | AGCTCCAACATCGGAAAAAATATT |
| (NeuB2-80) | 149) | |
| 80LC-CDR2-DNA | | AATAATAGT |
| (NeuB2-80) | | |
| 80LC-CDR3-DNA | (SEQ ID NO: | GCAGCATGGGATGACAGCCTGAGAT |
| (NeuB2-80) | 150) | CTTGGCTG |
| 93LC-CDR1-DNA | (SEQ ID NO: | AGCAGTGACGTTGGTGGTTATAACT |
| (NeuB2-93) | 151) | AT |
| 93LC-CDR2-DNA | | GAGGTCAGT |
| (NeuB2-93) | | |
| 93LC-CDR3-DNA | (SEQ ID NO: | AGCTCATATACAAGCAGCAGCACTC |
| (NeuB2-93) | 152) | CTTGGGTG |
| 110LC-CDR1-DNA | (SEQ ID NO: | AGCAGTGACGTTGGTGGTTATAACT |
| (NeuB2-110) | 153) | AT |
| 110LC-CDR2-DNA | | GAGGTCACT |
| (NeuB2-110) | | |
| 110LC-CDR3-DNA | (SEQ ID NO: | AGCTCATATACAAGCAGCAGCACCC |
| (NeuB2-110) | 154) | CGTATGTC |
| 128LC-CDR1-DNA | (SEQ ID NO: 155) | CAGGACATTAGCAACTAT |
| (NeuB2-128) | | |
| 128LC-CDR2-DNA | | GATGCATCC |
| (NeuB2-128) | | |
| 128LC-CDR3- | (SEQ ID NO: | CAACAGTATGATAATCTCCCCCTCA |
| DNAA | 156) | CT |
| (NeuB2-128) | | |

TABLE 12
DNA variable sequences of antibody heavy (H) chains

| Sequence | Sequence | |
|---|---|---|
| Label | ID | DNA Sequence |
| 8H-DNA | (SEQ ID | CAGCTGCAGCTGCAGGAGTCAGGTCCAGGACTGGTGAAG |
| | NO: 157) | CCCTCGCAGACCCTCTCACTCACCTGTGCCATCTCCGGGG |
| | | GCAATGTCTCTAGTAAAAGTGCTGCTTGGAACTGGATCA |
| | | GGCAGTCCCCATCGCGAGGCCTTGAGTGGCTGGGAAGGA |
| | | CATACTACAGGTCCAAGTGGTATAACGATTATGCAGTCTC |
| | | TGTGAAAAGTCGAATAACCATCAACCCAGACACATCCAA |
| | | GAACCAGTTCTCCCTGCAGTTGAAGTCTGTGACTCCCGAG |
| | | GACACGGCTGTGTATTACTGTGCAAGAAGCCTGGGTGAG |
| | | GGCTGGTTCCCATACTTCTACGGTATGGACGTCTGGGGCC |
| | | AAGGGACCACGGTCACCGTCTCCTCA |
| 9H-DNA | (SEQ ID | CAGGTCCAGCTTGTGCAGTCTGGGGCTGAGGTGAAGAAG |
| | NO: 158) | CCTGGGGCCTCAGTGAAGGTTTCCTGCAAGGCTTCTGGAT |
| | | ACACCTTCACTAGCTATGCTATGCATTGGGTGCGCCAGGC |
| | | CCCCGGACAAAGGCTTGAGTGGATGGGATGGATCAACGC |
| | | TGGCAATGGTAACACAAAATATTCACAGAAGTTCCAGGG |
| | | CAGAGTCACCATTACCAGGGACACATCCGCGAGCACAGC |
| | | CTACATGGAGCTGAGCAGCCTGAGATCTGAAGACACGGC |
| | | TGTGTATTACTGTGCGAGAGATTTTATGGGATATTGTAGT |
| | | AGTACCAGCTGCTATGGGGTTACTGGTGCTTTTGATATCT |
| | | GGGGCCAAGGGACAATGGTCACTGTCTCCTCA |
| 13H- | (SEQ ID | CAGGTGCAGCTGGTGCAGTCTGGGGCTGAGGTGAAGAAG |
| DNA | NO: 159) | CCTGGGGCCTCAGTGAAGGTTTCCTGCAAGGCATCTGGAT |
| | | ACACCTTCACCAGCTACTATATGCACTGGGTGCGACAGG |
| | | CCCCTGGACAAGGGCTTGAGTGGATGGGAATAATCAACC |
| | | CTAGTGGTGGTAGCACAAGCTACGCACAGAAGTTCCAGG |
| | | GCAGAGTCACCATGACCAGGGACACGTCCACGAGCACAG |
| | | TCTACATGGAGCTGAGCAGCCTGAGATCTGAGGACACGG |
| | | CCGTGTATTACTGTGCGAGAGAGGATTGGGGTCCCTTTGA |
| | | CTACTGGGGCCAGGGGACCACGGTCACCGTCTCTTCA |
| 19H- | (SEQ ID | CAGGTGCAGCTGGTGGAGTCTGGGGGAGGTGTGGTACGG |
| DNA | NO: 160) | CCTGGGGGGTCCCTGAGACTCTCCTGTGCAGCCTCTGGAT |
| | | TCACCTTTGATGATTATGGCATGAGCTGGGTCCGCCAAGC |
| | | TCCAGGGAAGGGGCTGGAGTGGGTCTCTGGTATTAATTG |
| | | GAATGGTGGTAGCACAGGTTATGCAGACTCTGTGAAGGG |
| | | CCGATTCACCATCTCCAGAGACAACGCCAAGAACTCCCT |
| | | GTATCTGCAAATGAACAGTCTGAGAGCCGAGGACACGGC |
| | | CTTGTATTACTGTGCGAGAGAGGGGGACGGTGCTTTTGAT |
| | | ATCTGGGGCCAAGGAACCCTGGTCACCGTCTCCTCA |
| 29H- | (SEQ ID | CAGGTGCAGCTGGTGGAGACTGGGGGAGGCGTGGTCCAG |
| DNA | NO: 161) | CCTGGAAGGTCCCTGAGACTCTCGTGTGCAGCCTCTGGAT |
| | | TCACCTTCAGTGACTATGCTATGCACTGGGTCCGCCAGGC |
| | | TCCAGGCAAGGGGCTGGAGTGGGTGACACTTATATCATA |
| | | TGATGGAAGTCAAAAATATTACGCAGACTCCGTGAAGGG |
| | | CCGATTCACCATCTCCAGAGACGATTCCAAGAACACGGT |
| | | GGATCTGCAAATGAACAGCCTGAAAGCTGAGGACACGGC |
| | | TGTGTATTACTGTGCGAAGTTTCCGTCCTTAAATGACAAC |
| | | GTGGGTTTTGATGTCTGGGGCCAGGGGACAATGGTCACC |
| | | GTCTCCTCA |
| 37H- | (SEQ ID | CAGGTGCAGTTGATGCAGTCTGGGGCTGAGGTGAAGAAG |
| DNA | NO: 162) | CCTGGGTCCTCGGTGAAGGTCTCCTGCAAGGCTTCTGGAG |
| | | GCACCTTCAGCAGCTATGCTATCAGCTGGGTGCGACAGG |
| | | CCCCTGGACAAGGGCTTGAGTGGATGGGAGGGATCATCC |
| | | CTATCTTTGGTACAGCAAACTACGCACAGAAGTTCCAGG |
| | | GCAGAGTCACGATTACCGCGGACGAATCCACGAGCACAG |
| | | CCTACATGGAGCTGAGCAGCCTGAGATCTGAGGACACGG |
| | | CCGTGTATTACTGTGCGAGAGGTAACCACGTATTACGATT |
| | | TTTGGAGTGGTTACCCAACTGGTTCGACCCCTGGGGCCAA |
| | | GGAACCCTGGTCACCGTCTCCTCA |
| 40H- | (SEQ ID | CAGGTTCAGCTTGTGCAGTCTGGAGCTGAGGTGAAGAAG |
| DNA | NO: 163) | CCTGGGGCCTCAGTGAAGGTCTCCTGCAAGGCTTCTGGTT |
| | | ACACCTTTACCAACTATGGTATCAGTTGGGTGCGACAAGC |
| | | CCCGGGACAAGGGCTTGAGTGGATGGGCTGGATCAGCGG |
| | | TGACGCTGGTGACACAAAATTTGCACAGAAGTTCCAGGG |
| | | CAGAGTCACCATGACGACAGACACATCCACGACTACAAC |
| | | GTACATGGAGCTGAGGAGCCTGAGATCTGACGACACGGC |
| | | CGTGTATTACTGTGCGAGAGATGGGGAATATATTCCTATG |
| | | TTTCGGGGATTTGACAACTACTACGGTCTGGACGTCTGGG |
| | | GCCAAGGGACCACGGTCACTGTCTCCTCA |
| 55H- | (SEQ ID | GAAGTGCAGCTGGTGCAGTCTGGGGCTGAGGTGAAGAAG |
| DNA | NO: 164) | CCTGGGTCCTCGGTGAAGGTCTCCTGCAAGGCTTCTGGAG |
| | | GCACCTTCAGCAGCTATGCTATCAGCTGGGTGCGACAGG |
| | | CCCCTGGACAAGGGCTTGAGTGGATGGGAGGGATCATCC |
| | | CTATCTTTGGTACAGCAAACTACGCACAGAAGTTCCAGG |
| | | GCAGAGTCACGATTACCGCGGACGAATCCACGAGCACAG |
| | | CCTACATGGAGCTGAGCAGCCTGAGATCTGAGGACACGG |
| | | CCGTGTATTACTGTGCGAGAGGCGTGGACCAGGTTGACT |
| | | ACTGGGGCCAGGGAACCCTGGTCACCGTCTCCTCA |
| 60H- | (SEQ ID | CAGGTGCAGCTACAGGAGTCGGGCCCAGGACTGGTGAAG |
| DNA | NO: 165) | CCTTCGGGGACCCTATCCCTCACCTGCGCTGTCTCTGGTG |
| | | GCTCCATCACCAGTAGTAACTGGTGGAGTTGGGTCCGCC |
| | | AGCCCCCAGGGAAGGGGCTGGAGTGGATTGGCGATATCT |
| | | CTCATAGTGGGAGCACCAACTACAACCCGTCCCTCAAGG |
| | | GTCGAGTCACCATGTCAGTGGACAGGTCCAAGAACCAGT |
| | | TCTTCCTGAAGCTGAGTTCTGTGACCGCCGCGGACACGGC |
| | | CATGTATTACTGTGCGAGAGCCTTTCACGGTATGGACGTC |
| | | TGGGGCCAAGGGACCACGGTCACCGTCTCTTCA |
| 80H- | (SEQ ID | CAGGTTCAGCTTGTGCAGTCTGGAGCTGAGGTGAAGAAG |
| DNA | NO: 166) | CCTGGGGCCTCAGTGAAGGTCTCCTGCAAGGCTTCTGGTT |
| | | ACACCTTTACCAGCTATGGTATCAGCTGGGTGCGACAGG |
| | | CCCCTGGACAAGGGCTTGAGTGGATGGGATGGATCAGCG |
| | | CTTACAATGGTAACACAAACTATGCACAGAAGCTCCAGG |
| | | GCAGAGTCACCATGACCACAGACACATCCACGAGCACAG |
| | | CCTACATGGAGCTGAGGAGCCTGAGATCTGACGACACGG |
| | | CCGTGTATTACTGTGCGAGAGATGGGGGTGGCGGGTACT |
| | | TTGACTACTGGGGCCAGGGAACCCTGGTCACCGTCTCCTC |
| | | A |
| 93H- | (SEQ ID | CAGGTGCAGTTGATGCAGTCTGGGGCTGAGGTGAAGAAG |
| DNA | NO: 167) | CCTGGGTCCTCGGTGAAGGTCTCCTGCAAGGCTTCTGGAG |
| | | GCAGGTTCAGCAGCAATGTTATGAGCTGGGTGCGACAGG |
| | | CCCCTGGACAAGGGCTTGAGTGGATGGGAGGGATCATCC |
| | | CTATTTTTGGTACACCAAAGTACGCACAGAAGTTCCAGG |
| | | GCAGAGTCACGATTACCGCGGACAAATCCACGAGCACAG |
| | | CCTACATGGAGCTGAGCAGCCTGAGATCTGAGGACACGG |
| | | CCGTGTATTACTGTGCGAGCGGTAGTACGCCTTTTGATGC |
| | | TTTTGATATCTGGGGCCAAGGGACAATGGTCACCGTCTCT |
| | | TCA |
| 110H- | (SEQ ID | CAGGTGCAGCTGGTGGAGTCTGGGGGAGGCTTGGTCAAG |
| DNA | NO: 168) | CCTGGAGGGTCCCTGAGACTCTCCTGTGGAATTTCTGGAT |
| | | TCACCTTCAGTGACTACTATATGAGCTGGATCCGCCAGGC |
| | | TCCAGGGAAGGGGCTGGAGTGGGTTTCATTCATTGGTACT |
| | | AGTGGTAGTACCATATACTACGCAGACTCTGTGAAGGGC |
| | | CGATTCACCATCTCCAGGGACAACGCCAATAACTCACTGT |
| | | TTCTTCAAATGAACAGCCTGACAGTCGAAGACACGGCCG |
| | | TGTATTACTGTGCGAGAGAGGCCACCTCTGGAACCTACTT |
| | | TGACTACTGGGGCCAGGGAACCCTGGTCAGCGTCTCCTC |
| | | A |
| 128H- | (SEQ ID | CAGGTGCAGCTACAGCAGTCTGGGGCTGAGGTGAAGAAG |
| DNA | NO: 169) | CCTGGGTCCTCGGTGAAGGTCTCCTGCAAGGCTTCTGGAG |
| | | GCACCTTCAGCAGCTATGCTATCAGCTGGGTGCGACAGG |
| | | CCCCTGGACAAGGGCTTGAGTGGATGGGAGGGATCATCC |
| | | CTATCTTTGGTACAGCAAACTACACACAGAAGTTCCAGG |
| | | GCAGAGTCACGATTACCGCGGACGAATCCACGAGCACAG |
| | | CCTACATGGAGCTGAGCAGCCTGAGATCTGAGGACACGG |
| | | CCGTGTATTACTGTGCGAGAGGTCCGAGTGGTAGCCCTTA |
| | | CGGTATGGACGTCTGGGGCCAAGGGACCACGGTCATCGT |
| | | CTCCTCA |

TABLE 13
DNA variable sequences of antibody light (L) chains

| Sequence | Sequence | |
|---|---|---|
| Label | ID | DNA Sequence |
| 8L-DNA | (SEQ ID | CAGTCTGTGCTGACTCAGCCTGCCTCCGTGTCAGGGTCTCCT |
| | NO: 170) | GGACAGTCGATCACCATCTCCTGCACTGGGACCAACAGAGA |
| | | CGTTGGTGGTTATGACTATGTCTCCTGGTACCAACAACACCC |
| | | AGGCAAAGCCCCCAAACTCATGATTTATGGTGTCAGTAATCG |
| | | GCCCTCAGGGATTTCTAATCGCTTCTCTGGCTCCAAGTCTGG |
| | | CAGCACGGCCTCCCTGACCATCTCTGGCCTTCAGGTTGAGGA |
| | | CGAGGCTGCTTATTACTGCTCTTCATACACAGCCAGCCGCAC |
| | | TGTGGTGTTCGGCGGAGGGACCAAGGTCACCGTCCTA |
| 9L-DNA | (SEQ ID | CAGTCTGTGCTGACTCAGCCACCCTCAGCGTCTGGGACCCCC |
| | NO: 171) | GGGCAGAGGGTCACCATCTCTTGTTCTGGAAGCAGCTCCAGC |
| | | ATCGGAAGTAATCCTGTAAACTGGTACCAGCAGCTCCCAGG |
| | | AACGGCCCCCAAACTCCTCATCTATAGTAATAATCAGCGGCC |
| | | CTCAGGGGTCCCTGACCGATTCTCTGGCTCCAAGTCTGGCAC |
| | | CTCACCCTCCCTGGCCATCAGTGGGCTCCAGTCTGAGGATGA |
| | | GGCTGATTATTACTGTGCAGCATGGGATGACAGCCTGAATGG |
| | | CGATTATGTCTTCGGAACTGGGACCAAGGTCACCGTCCTA |
| 13L- | (SEQ ID | GACATCCAGATGACCCAGTCTCCATCCTCCCTGTCTGCATCT |
| DNA | NO: 172) | GTAGGAGACAGAGTCACCATCACTTGCCGGGCAAGTCAGAG |
| | | CATAAGCAACTTTGTAAATTGGTATCAGCAGAAACCAGGGG |
| | | AAGTCCCTAAGCTCCTGATCTACGGTGCATCCAGTTTGCAGA |
| | | GTGGGGTCCCATCAAGGTTCAGTGGCAGTGGATCTGGGACG |
| | | GATTTCACTCTCACCATCAGCAGTCTGCAACCTGAAGATTTT |
| | | GCGACTTACTACTGTCAACAGGGTTACAGTGTCCCACTCACT |
| | | TTCGGCGGAGGGACCAAGGTGGAAATCAAA |
| 19L- | (SEQ ID | CAGGCTGTGGTGACTCAGCCACCCTCAGCGTCTGGGACCCCC |
| DNA | NO: 173) | GGGCAGAGGGTCACCATCTCTTGTTCTGGAAGCGGCTCCAAC |
| | | ATCAGAAGTAATGGTGTAACTTGGTACCAGCAGTTCCCAGG |
| | | AACGGCCCCCAAACTCCTCATTTATGATAATAATCAGCGGCC |
| | | CTCAGGGGTCCCTGGCCGATTCTCTGGCTCCAAGTCTGGCAC |
| | | CTCAGGCTCCCTGGCCATCAGTGGACTCCAGTCTGAGGATGA |
| | | GGCTGATTATTACTGTGCAGCATGGGATGACAACCTGAATGC |
| | | TTGGGTGTTCGGCGCAGGGACCCAGCTGACCGTCCTA |
| 29L- | (SEQ ID | CAGTCTGTGCTGACGCAGCCACCCTCGGTGTCTGGAGCCCCC |
| DNA | NO: 174) | AGGGAGAGGGTCACCATCTCCTGTTCTGGAAGCACCTCCAAT |
| | | ATCGAAAATAATGCTGTCAACTGGTACCAGCAGCTCCCAGG |
| | | CAAGGCTCCCAAACTCCTCATCTATTATGATGATCTAGTGCC |
| | | CTCAGGGGTCTCTGACCGATTCTCTGGTTCCAAGTCCGGCAC |
| | | CTCAGCCTCCCTGGCCATCAGTGGGCTCCAGTCTGGTGATGA |
| | | GGCTTATTATTACTGTGCAGTTTGGGATGACAGCCTGAATGG |
| | | TCCGGTTTTCGGCGGAGGGACCAAGGTCACCGTCCTA |
| 37L- | (SEQ ID | CAGTCTGTGCTGACTCAGCCACCCTCAGCGTCTGGGACCCCC |
| DNA | NO: 175) | GGGCAGAGGGTCACCATCTCTTGTTCTGGAAGCAGCTCCAAC |
| | | ATCGGAAGTAATACTGTAAACTGGTACCAGCAGTTCCCAGG |
| | | AACGGCCCCCAAACTCCTCATCTATAGTAATAATCAGCGGCC |
| | | CTCAGGGGTCCCTGACCGATTCTCTGGCTCCAAGTCTGGCAC |
| | | CTCAGCCTCCCTGGCCATCAGTGGGCTCCAGTCTGAGGATGA |
| | | GGCTGATTATTACTGTGCAGCATGGGATGACAGCCTGAATGG |
| | | TCATGTCTTCGGAACTGGGACCAAGGTCACCGTCCTA |
| 40L- | (SEQ ID | CAGCCTGTGCTGACTCAGCCACCCTCGGTGTCTGAGGCCCCC |
| DNA | NO: 176) | AGGCAGAGGGTCACCATCTCCTGTTCTGGAAGCAGCTCCAAC |
| | | ATCGGAAAGAATGCTGTAAACTGGTATCAGCAATTCCCAGG |
| | | AAAGGCTCCCAAACTCCTCATCTATTATGATGATCTACTGCC |
| | | CTCGGGGGTCTCTGACCGATTCTCTGGCTCCAGGTCTGGCAC |
| | | CTCAGCCTCCCTGGCCATCAGTGGACTCCAGTCTGAGGATGA |
| | | GGCTGATTATTACTGTGCAGCATGGGATGACAGACTGAAAG |
| | | GATACGTCTTCGGAACTGGGACCCAGCTGACCGTCCTA |
| 55L- | (SEQ ID | CAGGCTGTGGTGACTCAGCCTGCCTCCGTGTCTGGGTCTCCT |
| DNA | NO: 177) | GGACAGTCGATCACCATCTCCTGCACTGGAACCAGCAGTGA |
| | | CGTTGGTGGTTATAACTATGTCTCCTGGTACCAACAGCACCC |
| | | AGGCAAAGCCCCCAAACTCATGATTTATGATGTCAGTAATCG |
| | | GCCCTCAGGGGTTTCTAATCGCTTCTCTGGCTCCAAGTCTGG |
| | | CAACACGGCCTCCCTGACCATCTCTGGGCTCCAGGCTGAGGA |
| | | CGAGGCTGATTATTACTGCAGCTCATATACAAGCAGCAGCAC |
| | | CCCTTATGTCTTCGGAACTGGGACCAAGGTCACCGTCCTA |
| 60L- | (SEQ ID | CAGTCTGTGCTGACTCAGCCACCCTCGGTGTCTGAAGCCCCC |
| DNA | NO: 178) | AGGCAGAGGGTCACCATCTCCTGTTCTGGAAGCAGCTCCAAT |
| | | ATCGCAAATAATGCTGTAAACTGGTACCAGCAGCTCCCAGG |
| | | AAAGGCTCCCAAACTCCTCATTTATTATGATGATCTGTTGGC |
| | | CTCAGGGGTCTCTGACCGATTTTCTGGCTCCAGGTCTGGCAC |
| | | CTCAGCCGCCCTGGCCATCAGTGGCCTCCAGTCTGAGGATGA |
| | | GGCTGATTATTACTGTGCAACATGGGATGACGACCTGAATGG |
| | | TCCGATATTCGGCGGAGGGACCAAGGTCACCGTCCTA |
| 80L- | (SEQ ID | CAGGCTGTGGTGACTCAGCCACCCTCAGCGTCTGGGACCCCC |
| DNA | NO: 179) | GGGCAGAGGATCACCATCTCTTGTTCTGGAAGCAGCTCCAAC |
| | | ATCGGAAAAAATATTGTAAACTGGTACCAGCAGGTCCCAGG |
| | | AGCGGCCCCCAAACTCCTCATCTATAATAATAGTCAGCGGCC |
| | | CTCAGGGGTCCCTGACCGATTCTCTGGCTCCAAGTCTGGCAC |
| | | CTCAGCCTCCCTGGCCATCAGTGGGCTCCAGTCTGAGGATGA |
| | | GGCTGATTATTACTGTGCAGCATGGGATGACAGCCTGAGATC |
| | | TTGGCTGTTCGGCGGAGGGACCAAGGTCACCGTCCTA |
| 93L- | (SEQ ID | CAGCCTGTGCTGACTCAGCCTGCCTCCGTGTCTGGGTCTCCT |
| DNA | NO: 180) | GGACAGTCGATCACCATCTCCTGCACTGGAACCAGCAGTGA |
| | | CGTTGGTGGTTATAACTATGTCTCCTGGTACCAACAGCACCC |
| | | AGGCAAAGCCCCCAAACTCATGATTTATGAGGTCAGTAATC |
| | | GGCCCTCAGGGGTTTCTAATCGCTTCTCTGGCTCCAAGTCTG |
| | | GCAACACGGCCTCCCTGACCATCTCTGGGCTCCAGGCTGAGG |
| | | ACGAGGCTGATTATTACTGCAGCTCATATACAAGCAGCAGC |
| | | ACTCCTTGGGTGTTCGGCGGAGGGACCAAGGTCACCGTCCTA |
| 110L- | (SEQ ID | CAGGCTGTGGTGACTCAGCCTGCCTCCGTGTCTGGGTCTCCT |
| DNA | NO: 181) | GGACAGTCGATCACCATCTCCTGCACTGGAACCAGCAGTGA |
| | | CGTTGGTGGTTATAACTATGTCTCCTGGTACCAACAGCACCC |
| | | AGGCAAAGCCCCCAAACTCATGATTTATGAGGTCACTAATCG |
| | | GCCCTCAGGGGTTTCTAATCGCTTCTCTGGCTCCAAGTCTGG |
| | | CAACACGGCCTCCCTGACCATCTCTGGGCTCCAGGCTGAGGA |
| | | CGAGGCTGATTATTACTGCAGCTCATATACAAGCAGCAGCAC |
| | | CCCGTATGTCTTCGGAACTGGGACCAAGGTCACCGTCCTA |
| 128L- | (SEQ ID | GACATCCAGATGACCCAGTCTCCATCCTCCCTGTCTGCATCT |
| DNA | NO: 182) | GTAGGAGACAGAGTCACCATCACTTGCCAGGCGAGTCAGGA |
| | | CATTAGCAACTATTTAAATTGGTATCAGCAGAAACCAGGGA |
| | | AAGCCCCTAAGCTCCTGATCTACGATGCATCCAATTTGGAAA |
| | | CAGGGGTCCCATCAAGGTTCAGTGGAAGTGGATCTGGGACA |
| | | GATTTTACTTTCACCATCAGCAGCCTGCAGCCTGAAGATATT |
| | | GCAACATATTACTGTCAACAGTATGATAATCTCCCCCTCACT |
| | | TTCGGCGGAGGGACCAAAGTGGATATCAAA |

TABLE 14
Heavy and Light Chain Pairs of NeuB2 mAbs

| mAb Name | Heavy chain | light chain |
|---|---|---|
| NeuB2-8 | 8-HC | 8-LC |
| NeuB2-9 | 9-HC | 9-LC |
| NeuB2-13 | 13-HC | 13-LC |
| NeuB2-19 | 19-HC | 19-LC |
| NeuB2-29 | 29-HC | 29-LC |
| NeuB2-37 | 37-HC | 37-LC |
| NeuB2-40 | 40-HC | 40-LC |
| NeuB2-55 | 55-HC | 55-LC |
| NeuB2-60 | 60-HC | 60-LC |
| NeuB2-80 | 80-HC | 80-LC |
| NeuB2-93 | 93-HC | 93-LC |
| NeuB2-110 | 110-HC | 110-LC |
| NeuB2-128 | 128-HC | 128-LC |

### List of Embodiments

1. An isolated monoclonal antibody, wherein the antibody specifically binds to LILRB2 and wherein the antibody competes for binding of the LILRB2 epitopes with a NeuB2-8, NeuB2-9, NeuB2-13, NeuB2-19, NeuB2-29, NeuB2-37, NeuB2-40, NeuB2-55, NeuB2-60, NeuB2-80, NeuB2-93, NeuB2-110, or NeuB2-128 monoclonal antibody.
2. The antibody of Embodiment 1, or antigen-binding fragment thereof, wherein the antibody comprises:
   (a) a first V_{H} CDR at least 80% identical to the V_{H} CDR1 (SEQ ID NO: 1) of NeuB2-8, at least 80% identical to V_{H} CDR1 (SEQ ID NO: 4) of NeuB2-9, at least 80% identical to the V_{H} CDR1 (SEQ ID NO: 7) of NeuB2-13, at least 80% identical to the V_{H} CDR1 (SEQ ID NO: 10) of NeuB2-19, at least 80% identical to the V_{H} CDR1 (SEQ ID NO: 13) of NeuB2-29, at least 80% identical to the V_{H} CDR1 (SEQ ID NO: 16) of NeuB2-37, at least 80% identical to the V_{H} CDR1 (SEQ ID NO: 19) of NeuB2-40, at least 80% identical to the V_{H} CDR1 (SEQ ID NO: 22) of NeuB2-55, at least 80% identical to the V_{H} CDR1 (SEQ ID NO: 25) of NeuB2-60, at least 80% identical to the V_{H} CDR1 (SEQ ID NO: 28) of NeuB2-80, at least 80% identical to the V_{H} CDR1 (SEQ ID NO: 31) of NeuB2-93, at least 80% identical to the V_{H} CDR1 (SEQ ID NO: 34) of NeuB2-110, or at least 80% identical to the V_{H} CDR1 (SEQ ID NO: 37) of NeuB2-128;
   (b) a second V_{H} CDR at least 80% identical to the V_{H} CDR2 (SEQ ID NO: 2) of NeuB2-8, at least 80% identical to the V_{H} CDR2 (SEQ ID NO: 5), at least 80% identical to the V_{H} CDR2 (SEQ ID NO: 8), at least 80% identical to the V_{H} CDR2 (SEQ ID NO: 11), at least 80% identical to the V_{H} CDR2 (SEQ ID NO: 14), at least 80% identical to the V_{H} CDR2 (SEQ ID NO: 17), at least 80% identical to the V_{H} CDR2 (SEQ ID NO: 20), at least 80% identical to the V_{H} CDR2 (SEQ ID NO: 23), at least 80% identical to the V_{H} CDR2 (SEQ ID NO: 26), at least 80% identical to the V_{H} CDR2 (SEQ ID NO: 29), at least 80% identical to the V_{H} CDR2 (SEQ ID NO: 32), at least 80% identical to the V_{H} CDR2 (SEQ ID NO: 35), or at least 80% identical to the V_{H} CDR2 (SEQ ID NO: 38);
   (c) a third V_{H} CDR at least 80% identical to V_{H} CDR3 (SEQ ID NO: 3) of NeuB2-8, at least 80% identical to V_{H} CDR3 (SEQ ID NO: 6) of NeuB2-9, at least 80% identical to V_{H} CDR3 (SEQ ID NO: 9) of NeuB2-13, at least 80% identical to V_{H} CDR3 (SEQ ID NO: 12) of NeuB2-19, at least 80% identical to V_{H} CDR3 (SEQ ID NO: 15) of NeuB2-29, at least 80% identical to V_{H} CDR3 (SEQ ID NO: 18) of NeuB2-37, at least 80% identical to V_{H} CDR3 (SEQ ID NO: 21) of NeuB2-40, at least 80% identical to V_{H} CDR3 (SEQ ID NO: 24) of NeuB2-55, at least 80% identical to V_{H} CDR3 (SEQ ID NO: 27) of NeuB2-60, at least 80% identical to V_{H} CDR3 (SEQ ID NO: 30) of NeuB2-80, at least 80% identical to V_{H} CDR3 (SEQ ID NO: 33) of NeuB2-93, at least 80% identical to V_{H} CDR3 (SEQ ID NO: 36) of NeuB2-110, or at least 80% identical to V_{H} CDR3 (SEQ ID NO: 39) of NeuB2-128;
   (d) a first V_{L} CDR at least 80% identical to V_{L} CDR1 (SEQ ID NO: 40) of NeuB2-8, at least 80% identical to V_{L} CDR1 (SEQ ID NO: 42) of NeuB2-9, at least 80% identical to V_{L} CDR1 (SEQ ID NO: 44) of NeuB2-13, at least 80% identical to V_{L} CDR1 (SEQ ID NO: 46) of NeuB2-19, at least 80% identical to V_{L} CDR1 (SEQ ID NO: 48) of NeuB2-29, at least 80% identical to V_{L} CDR1 (SEQ ID NO: 50) of NeuB2-37, at least 80% identical to V_{L} CDR1 (SEQ ID NO: 52) of NeuB2-40, at least 80% identical to V_{L} CDR1 (SEQ ID NO: 54) of NeuB2-55, at least 80% identical to V_{L} CDR1 (SEQ ID NO: 56) of NeuB2-60, at least 80% identical to V_{L} CDR1 (SEQ ID NO: 58) of NeuB2-80, at least 80% identical to V_{L} CDR1 (SEQ ID NO: 60) of NeuB2-93, at least 80% identical to V_{L} CDR1 (SEQ ID NO: 62) of NeuB2-110, or at least 80% identical to V_{L} CDR1 (SEQ ID NO: 64) of NeuB2-128;
   (e) a second V_{L} CDR at least 80% identical to V_{L} CDR2 (tripeptide GVS) of NeuB2-8, at least 80% identical to V_{L} CDR2 (tripeptide SNN) of NeuB2-9, at least 80% identical to V_{L} CDR2 (tripeptide GAS) of NeuB2-13, at least 80% identical to V_{L} CDR2 (tripeptide DNN) of NeuB2-19, at least 80% identical to V_{L} CDR2 (tripeptide YDD) of NeuB2-29, at least 80% identical to V_{L} CDR2 (tripeptide SNN) of NeuB2-37, at least 80% identical to V_{L} CDR2 (tripeptide YDD) of NeuB2-40, at least 80% identical to V_{L} CDR2 (Tripeptide DVS) of NeuB2-55, at least 80% identical to V_{L} CDR2 (Tripeptide YDD) of NeuB2-60, at least 80% identical to V_{L} CDR2 (Tripeptide NNS) of NeuB2-80, at least 80% identical to V_{L} CDR2 (Tripeptide EVS) of NeuB2-93, at least 80% identical to V_{L} CDR2 (Tripeptide EVS) of NeuB2-110, or at least 80% identical to V_{L} CDR2 (Tripeptide DAS) of NeuB2-128; and
   (f) a third V_{L} CDR at least 80% identical to V_{L} CDR3 (SEQ ID NO: 41) of NeuB2-8, at least 80% identical to V_{L} CDR3 (SEQ ID NO: 43) of NeuB2-9, at least 80% identical to V_{L} CDR3 (SEQ ID NO: 45) of NeuB2-13, at least 80% identical to V_{L} CDR3 (SEQ ID NO: 47) of NeuB2-19, at least 80% identical to V_{L} CDR3 (SEQ ID NO: 80) of NeuB2-29, at least 80% identical to V_{L} CDR3 (SEQ ID NO: 51) of NeuB2-37, at least 80% identical to V_{L} CDR3 (SEQ ID NO: 53) of NeuB2-40, at least 80% identical to V_{L} CDR3 (SEQ ID NO: 55) of NeuB2-55, at least 80% identical to V_{L} CDR3 (SEQ ID NO: 57) of NeuB2-60, at least 80% identical to V_{L} CDR3 (SEQ ID NO: 59) of NeuB2-80, at least 80% identical to V_{L} CDR3 (SEQ ID NO: 61) of NeuB2-93, at least 80% identical to V_{L} CDR3 (SEQ ID NO: 63) of NeuB2-110, or at least 80% identical to V_{L} CDR3 (SEQ ID NO: 65) of NeuB2-128.
3. The isolated antibody of Embodiment 2, wherein the antibody comprises:
   (a) a first V_{H} CDR is identical to SEQ ID NO: 1;
   (b) a second V_{H} CDR is identical to SEQ ID NO: 2;
   (c) a third V_{H} CDR is identical to SEQ ID NO: 3;
   (d) a first V_{L} CDR is identical to SEQ ID NO: 40;
   (e) a second V_{L} CDR is identical to tripeptide GVS; and
   (f) a third V_{L} CDR is identical to SEQ ID NO: 41.
4. The isolated antibody of Embodiment 2, wherein the antibody comprises:
   (a) a first V_{H} CDR is identical to SEQ ID NO: 4;
   (b) a second V_{H} CDR is identical to SEQ ID NO: 5;
   (c) a third V_{H} CDR is identical to SEQ ID NO: 6;
   (d) a first V_{L} CDR is identical to SEQ ID NO: 42;
   (e) a second V_{L} CDR is identical to tripeptide SNN; and
   (f) a third V_{L} CDR is identical to SEQ ID NO: 43.
5. The isolated antibody of Embodiment 2, wherein the antibody comprises:
   (a) a first V_{H} CDR is identical to SEQ ID NO: 7;
   (b) a second V_{H} CDR is identical to SEQ ID NO: 8;
   (c) a third V_{H} CDR is identical to SEQ ID NO: 9;
   (d) a first V_{L} CDR is identical to SEQ ID NO: 44;
   (e) a second V_{L} CDR is identical to tripeptide GAS; and
   (f) a third V_{L} CDR is identical to SEQ ID NO: 45.
6. The isolated antibody of Embodiment 2, wherein the antibody comprises:
   (a) a first V_{H} CDR is identical to SEQ ID NO: 10;
   (b) a second V_{H} CDR is identical to SEQ ID NO: 11;
   (c) a third V_{H} CDR is identical to SEQ ID NO: 12;
   (d) a first V_{L} CDR is identical to SEQ ID NO: 46;
   (e) a second V_{L} CDR is identical to tripeptide DNN; and
   (f) a third V_{L} CDR is identical to SEQ ID NO: 47.
7. The isolated antibody of Embodiment 2, wherein the antibody comprises:
   (a) a first V_{H} CDR is identical to SEQ ID NO: 13;
   (b) a second V_{H} CDR is identical to SEQ ID NO: 14;
   (c) a third V_{H} CDR is identical to SEQ ID NO: 15;
   (d) a first V_{L} CDR is identical to SEQ ID NO: 48;
   (e) a second V_{L} CDR is identical to tripeptide YDD; and
   (f) a third V_{L} CDR is identical to SEQ ID NO: 49.
8. The isolated antibody of Embodiment 2, wherein the antibody comprises:
   (a) a first V_{H} CDR is identical to SEQ ID NO: 16;
   (b) a second V_{H} CDR is identical to SEQ ID NO: 17;
   (c) a third V_{H} CDR is identical to SEQ ID NO: 18;
   (d) a first V_{L} CDR is identical to SEQ ID NO: 50;
   (e) a second V_{L} CDR is identical to tripeptide SNN; and
   (f) a third V_{L} CDR is identical to SEQ ID NO: 51.
9. The isolated antibody of Embodiment 2, wherein the antibody comprises:
   (a) a first V_{H} CDR is identical to SEQ ID NO: 19;
   (b) a second V_{H} CDR is identical to SEQ ID NO: 20;
   (c) a third V_{H} CDR is identical to SEQ ID NO: 21;
   (d) a first V_{L} CDR is identical to SEQ ID NO: 52;
   (e) a second V_{L} CDR is identical to tripeptide YDD; and
   (f) a third V_{L} CDR is identical to SEQ ID NO: 53.
10. The isolated antibody of Embodiment 2, wherein the antibody comprises:
   (a) a first V_{H} CDR is identical to SEQ ID NO: 22;
   (b) a second V_{H} CDR is identical to SEQ ID NO: 23;
   (c) a third V_{H} CDR is identical to SEQ ID NO: 24;
   (d) a first V_{L} CDR is identical to SEQ ID NO: 54;
   (e) a second V_{L} CDR is identical to Tripeptide DVS; and
   (f) a third V_{L} CDR is identical to SEQ ID NO: 55.
11. The isolated antibody of Embodiment 2, wherein the antibody comprises:
   (a) a first V_{H} CDR is identical to SEQ ID NO: 25;
   (b) a second V_{H} CDR is identical to SEQ ID NO: 26;
   (c) a third V_{H} CDR is identical to SEQ ID NO: 27;
   (d) a first V_{L} CDR is identical to SEQ ID NO: 56;
   (e) a second V_{L} CDR is identical to Tripeptide YDD; and
   (f) a third V_{L} CDR is identical to SEQ ID NO: 57.
12. The isolated antibody of Embodiment 2, wherein the antibody comprises:
   (a) a first V_{H} CDR is identical to SEQ ID NO: 28;
   (b) a second V_{H} CDR is identical to SEQ ID NO: 29;
   (c) a third V_{H} CDR is identical to SEQ ID NO: 30;
   (d) a first V_{L} CDR is identical to SEQ ID NO: 58;
   (e) a second V_{L} CDR is identical to Tripeptide NNS; and
   (f) a third V_{L} CDR is identical to SEQ ID NO: 59.
13. The isolated antibody of Embodiment 2, wherein the antibody comprises:
   (a) a first V_{H} CDR is identical to SEQ ID NO: 31;
   (b) a second V_{H} CDR is identical to SEQ ID NO: 32;
   (c) a third V_{H} CDR is identical to SEQ ID NO: 33;
   (d) a first V_{L} CDR is identical to SEQ ID NO: 60;
   (e) a second V_{L} CDR is identical to Tripeptide EVS; and
   (f) a third V_{L} CDR is identical to SEQ ID NO: 61.
14. The isolated antibody of Embodiment 2, wherein the antibody comprises:
   (a) a first V_{H} CDR is identical to SEQ ID NO: 34;
   (b) a second V_{H} CDR is identical to SEQ ID NO: 35;
   (c) a third V_{H} CDR is identical to SEQ ID NO: 36;
   (d) a first V_{L} CDR is identical to SEQ ID NO: 62;
   (e) a second V_{L} CDR is identical to Tripeptide EVS; and
   (f) a third V_{L} CDR is identical to SEQ ID NO: 63.
15. The isolated antibody of Embodiment 2, wherein the antibody comprises:
   (a) a first V_{H} CDR is identical to SEQ ID NO: 37;
   (b) a second V_{H} CDR is identical to SEQ ID NO: 38;
   (c) a third V_{H} CDR is identical to SEQ ID NO: 39;
   (d) a first V_{L} CDR is identical to SEQ ID NO: 64;
   (e) a second V_{L} CDR is identical to Tripeptide DAS; and
   (f) a third V_{L} CDR is identical to SEQ ID NO: 65.
16. The antibody of Embodiment 2, wherein the antibody comprises:
   (i) a V_{H} domain at least about 80% identical to the V_{H} domain (SEQ ID NO: 66) of NeuB2-8 or the humanized V_{H} domain of NeuB2-8 mAb; and a V_{L} domain at least about 80% identical to the V_{L} domain (SEQ ID NO: 79) of NeuB2-8 or the humanized V_{L} domain of NeuB2-8 mAb;
   (ii) a V_{H} domain at least about 80% identical to the V_{H} domain (SEQ ID NO: 67) of NeuB2-9 or the humanized V_{H} domain of NeuB2-9 mAb; and a V_{L} domain at least about 80% identical to the V_{L} domain (SEQ ID NO: 80) of NeuB2-9 or the humanized V_{L} domain of NeuB2-9 mAb;
   (iii) a V_{H} domain at least about 80% identical to the V_{H} domain (SEQ ID NO: 68) of NeuB2-13 or the humanized V_{H} domain of NeuB2-13 mAb; and a V_{L} domain at least about 80% identical to the V_{L} domain (SEQ ID NO: 81) of NeuB2-13 or the humanized V_{L} domain of NeuB2-13 mAb;
   (iv) a V_{H} domain at least about 80% identical to the V_{H} domain (SEQ ID NO: 69) of NeuB2-19 or the humanized V_{H} domain of NeuB2-19 mAb; and a V_{L} domain at least about 80% identical to the V_{L} domain (SEQ ID NO: 82) of NeuB2-19 or the humanized V_{L} domain of NeuB2-19 mAb;
   (v) a V_{H} domain at least about 80% identical to the V_{H} domain (SEQ ID NO: 70) of NeuB2-29 or the humanized V_{H} domain of NeuB2-29 mAb; and a V_{L} domain at least about 80% identical to the V_{L} domain (SEQ ID NO: 83) of NeuB2-29 or the humanized V_{L} domain of NeuB2-29 mAb;
   (vi) a V_{H} domain at least about 80% identical to the V_{H} domain (SEQ ID NO: 71) of NeuB2-37 or the humanized V_{H} domain of NeuB2-37 mAb; and a V_{L} domain at least about 80% identical to the V_{L} domain (SEQ ID NO: 84) of NeuB2-37 or the humanized V_{L} domain of NeuB2-37 mAb;
   (vii) a V_{H} domain at least about 80% identical to the V_{H} domain (SEQ ID NO: 72) of NeuB2-40 or the humanized V_{H} domain of NeuB2-40 mAb; and a V_{L} domain at least about 80% identical to the V_{L} domain (SEQ ID NO: 85) of NeuB2-40 or the humanized V_{L} domain of NeuB2-40 mAb;
   (viii) a V_{H} domain at least about 80% identical to the V_{H} domain (SEQ ID NO: 73) of NeuB2-55 or the humanized V_{H} domain of NeuB2-55 mAb; and a V_{L} domain at least about 80% identical to the V_{L} domain (SEQ ID NO: 86) of NeuB2-55 or the humanized V_{L} domain of NeuB2-55 mAb;
   (ix) a V_{H} domain at least about 80% identical to the V_{H} domain (SEQ ID NO: 74) of NeuB2-60 or the humanized V_{H} domain of NeuB2-60 mAb; and a V_{L} domain at least about 80% identical to the V_{L} domain (SEQ ID NO: 87) of NeuB2-60 or the humanized V_{L} domain of NeuB2-60 mAb;
   (x) a V_{H} domain at least about 80% identical to the V_{H} domain (SEQ ID NO: 75) of NeuB2-80 or the humanized V_{H} domain of NeuB2-80 mAb; and a V_{L} domain at least about 80% identical to the V_{L} domain (SEQ ID NO: 88) of NeuB2-80 or the humanized V_{L} domain of NeuB2-80 mAb;
   (xi) a V_{H} domain at least about 80% identical to the V_{H} domain (SEQ ID NO: 76) of NeuB2-93 or the humanized V_{H} domain of NeuB2-93 mAb; and a V_{L} domain at least about 80% identical to the V_{L} domain (SEQ ID NO: 89) of NeuB2-93 or the humanized V_{L} domain of NeuB2-93 mAb;
   (xii) a V_{H} domain at least about 80% identical to the V_{H} domain (SEQ ID NO: 77) of NeuB2-110 or the humanized V_{H} domain of NeuB2-110 mAb; and a V_{L} domain at least about 80% identical to the V_{L} domain (SEQ ID NO: 90) of NeuB2-110 or the humanized V_{L} domain of NeuB2-110 mAb, or
   (xiii) a V_{H} domain at least about 80% identical to the V_{H} domain (SEQ ID NO: 78) of NeuB2-128 or the humanized V_{H} domain of NeuB2-128 mAb; and a V_{L} domain at least about 80% identical to the V_{L} domain (SEQ ID NO: 91) of NeuB2-128 or the humanized V_{L} domain of NeuB2-128 mAb.
17. The antibody of Embodiment 16, wherein the antibody comprises a V_{H} domain (SEQ ID NO: 66) identical to the V_{H} domain of NeuB2-8 and a V_{L} domain (SEQ ID NO: 79) identical to the V_{L} domain of NeuB2-8.
18. The antibody of Embodiment 16, wherein the antibody comprises a V_{H} domain (SEQ ID NO: 67) identical to the V_{H} domain of NeuB2-9 and a V_{L} domain (SEQ ID NO: 80) identical to the V_{L} domain of NeuB2-9.
19. The antibody of Embodiment 16, wherein the antibody comprises a V_{H} domain (SEQ ID NO: 68) identical to the V_{H} domain of NeuB2-13 and a V_{L} domain (SEQ ID NO: 81) identical to the V_{L} domain of NeuB2-13.
20. The antibody of Embodiment 16, wherein the antibody comprises a V_{H} domain (SEQ ID NO: 69) identical to the V_{H} domain of NeuB2-19 and a V_{L} domain (SEQ ID NO: 82) identical to the V_{L} domain of NeuB2-19.
21. The antibody of Embodiment 16, wherein the antibody comprises a V_{H} domain (SEQ ID NO: 70) identical to the V_{H} domain of NeuB2-29 and a V_{L} domain (SEQ ID NO: 83) identical to the V_{L} domain of NeuB2-29.
22. The antibody of Embodiment 16, wherein the antibody comprises a V_{H} domain (SEQ ID NO: 71) identical to the V_{H} domain of NeuB2-37 and a V_{L} domain (SEQ ID NO: 84) identical to the V_{L} domain of NeuB2-37.
23. The antibody of Embodiment 16, wherein the antibody comprises a V_{H} domain (SEQ ID NO: 72) identical to the V_{H} domain of NeuB2-40 and a V_{L} domain (SEQ ID NO: 85) identical to the V_{L} domain of NeuB2-40.
24. The antibody of Embodiment 16, wherein the antibody comprises a V_{H} domain (SEQ ID NO: 73) identical to the V_{H} domain of NeuB2-55 and a V_{L} domain (SEQ ID NO: 86) identical to the V_{L} domain of NeuB2-55.
25. The antibody of Embodiment 16, wherein the antibody comprises a V_{H} domain (SEQ ID NO: 74) identical to the V_{H} domain of NeuB2-60 and a V_{L} domain (SEQ ID NO: 87) identical to the V_{L} domain of NeuB2-60.
26. The antibody of Embodiment 16, wherein the antibody comprises a V_{H} domain (SEQ ID NO: 75) identical to the V_{H} domain of NeuB2-80 and a V_{L} domain (SEQ ID NO: 88) identical to the V_{L} domain of NeuB2-80.
27. The antibody of Embodiment 16, wherein the antibody comprises a V_{H} domain (SEQ ID NO: 76) identical to the V_{H} domain of NeuB2-93 and a V_{L} domain (SEQ ID NO: 89) identical to the V_{L} domain of NeuB2-93.
28. The antibody of Embodiment 16, wherein the antibody comprises a V_{H} domain (SEQ ID NO: 77) identical to the V_{H} domain of NeuB2-110 and a V_{L} domain (SEQ ID NO: 90) identical to the V_{L} domain of NeuB2-110.
29. The antibody of Embodiment 16, wherein the antibody comprises a V_{H} domain (SEQ ID NO: 78) identical to the V_{H} domain of NeuB2-128 and a V_{L} domain (SEQ ID NO: 91) identical to the V_{L} domain of NeuB2-128.
30. The antibody of Embodiment 16, wherein the antibody is the NeuB2-8, NeuB2-9, NeuB2-13, NeuB2-19, NeuB2-29, NeuB2-37, NeuB2-40, NeuB2-55, NeuB2-60, NeuB2-80, NeuB2-93, NeuB2-110, or NeuB2-128 antibody
31. The antibody of any one of Embodiments 1-30, wherein the antibody is recombinant.
32. The antibody of any one of Embodiments 1-31, wherein the antibody is an IgG, IgM, IgA or an antigen binding fragment thereof.
33. The antibody of any one of Embodiments 1-30, wherein the antibody is a Fab′, a F(ab′)2, a F(ab′)3, a monovalent scFv, a bivalent scFv, or a single domain antibody.
34. The antibody of any one of Embodiments 1-16, wherein the antibody is a human, humanized antibody or de-immunized antibody.
35. The antibody of any one of Embodiments 1-30, wherein the antibody is conjugated to an imaging agent.
36. The antibody of any one of Embodiments 1-30, wherein the antibody inhibits or blocks LILRB2 interaction with ligands PS and oAβ as measured using an Octet 96-Red instrument or as measured using a LILRB2-chimeric cell reporter assay.
37. The antibody of any one of Embodiments 1-30, wherein the antibody rescues ligand-induced TREM2 signaling, as measured using a reporter cell assay.
38. A chimeric antigen receptor comprising an antigen-binding domain at least 80% identical to an antigen-binding domain of the monoclonal antibody of any one of the preceding Embodiments.
39. A composition comprising an antibody of any one of Embodiments 1-30 in a pharmaceutically acceptable carrier.
40. An isolated polynucleotide molecule comprising a nucleic acid sequence encoding an antibody of any one of Embodiments 1-30.
41. A recombinant polypeptide comprising an antibody V_{H} domain comprising CDRs 1-3 of the V_{H} domain of NeuB2-8 (SEQ ID NOs: 1, 2, and 3); CDRs 1-3 of the V_{H} domain of NeuB2-9 (SEQ ID NOs: 4, 5, and 6); CDRs 1-3 of the V_{H} domain of NeuB2-13 (SEQ ID NOs: 7, 8, and 9); CDRs 1-3 of the V_{H} domain of NeuB2-19 (SEQ ID NOs: 10, 11, and 12); CDRs 1-3 of the V_{H} domain of NeuB2-29 (SEQ ID NOs: 13, 14, and 15); CDRs 1-3 of the V_{H} domain of NeuB2-37 (SEQ ID NOs: 16, 17, and 18); CDRs 1-3 of the V_{H} domain of NeuB2-40 (SEQ ID NOs: 19, 20, and 21); CDRs 1-3 of the V_{H} domain of NeuB2-55 (SEQ ID NOs: 22, 23, and 24); CDRs 1-3 of the V_{H} domain of NeuB2-60 (SEQ ID NOs: 25, 26, and 27); CDRs 1-3 of the V_{H} domain of NeuB2-80 (SEQ ID NOs: 28, 29, and 30); CDRs 1-3 of the V_{H} domain of NeuB2-93 (SEQ ID NOs: 31, 32, and 33); CDRs 1-3 of the V_{H} domain of NeuB2-110 (SEQ ID NOs: 34, 35, and 36); or CDRs 1-3 of the V_{H} domain of NeuB2-128 (SEQ ID NOs: 37, 38, and 39).
42. A recombinant polypeptide comprising an antibody V_{L} domain comprising CDRs 1-3 of the V_{L} domain of NeuB2-8 (SEQ ID NO: 40, tripeptide GVS, and SEQ ID NO: 41); CDRs 1-3 of the V_{L} domain of NeuB2-9 (SEQ ID NO: 42, tripeptide SNN, and SEQ ID NO: 43); CDRs 1-3 of the V_{L} domain of NeuB2-13 (SEQ ID NO: 44, tripeptide GAS, and SEQ ID NO: 45); CDRs 1-3 of the V_{L} domain of NeuB2-19 (SEQ ID NO: 46, tripeptide DNN, and SEQ ID NO: 41); CDRs 1-3 of the V_{L} domain of NeuB2-29 (SEQ ID NO: 40, tripeptide YDD, and SEQ ID NO: 47); CDRs 1-3 of the V_{L} domain of NeuB2-37 (SEQ ID NO: 48, tripeptide SNN, and SEQ ID NO: 49); CDRs 1-3 of the V_{L} domain of NeuB2-40 (SEQ ID NO: 50, tripeptide YDD, and SEQ ID NO: 51); CDRs 1-3 of the V_{L} domain of NeuB2-55 (SEQ ID NO: 52, tripeptide DVS, and SEQ ID NO: 53); CDRs 1-3 of the V_{L} domain of NeuB2-60 (SEQ ID NO: 54, tripeptide YDD, and SEQ ID NO: 55); CDRs 1-3 of the V_{L} domain of NeuB2-80 (SEQ ID NO: 56, tripeptide NNS, and SEQ ID NO: 57); CDRs 1-3 of the V_{L} domain of NeuB2-93 (SEQ ID NO: 58, tripeptide EVS, and SEQ ID NO: 59); CDRs 1-3 of the V_{L} domain of NeuB2-110 (SEQ ID NO: 60, tripeptide EVT, and SEQ ID NO: 61); or CDRs 1-3 of the V_{L} domain of NeuB2-128 (SEQ ID NO: 62, tripeptide DAS, and SEQ ID NO: 63).
43. An isolated polynucleotide molecule comprising a nucleic acid sequence encoding a polypeptide of Embodiment 41 or 42.
44. A host cell comprising one or more polynucleotide molecule(s) encoding an antibody of any one of Embodiments 1-30 or a recombinant polypeptide of Embodiment 39 or 40.
45. The host cell of Embodiment 44, wherein the host cell is a mammalian cell, a yeast cell, a bacterial cell, a ciliate cell or an insect cell.
46. A method of manufacturing an antibody comprising:
   (a) expressing one or more polynucleotide molecule(s) encoding a V_{L} and V_{H} chain of an antibody of any one of Embodiments 1-30 in a cell; and
   (b) purifying the antibody from the cell.

The embodiments described above are presented by way of example only and are not intended as a limitation upon the concepts and principles of the present disclosure. As such, it will be appreciated by one having ordinary skill in the art that various changes in the elements and their configuration and arrangement are possible without departing from the spirit and scope of the present disclosure. Various features and aspects of the present disclosure are set forth in the following claims.

1. An isolated monoclonal antibody, wherein the antibody specifically binds to LILRB2 and wherein the antibody competes for binding of the LILRB2 epitopes with a NeuB2-8, NeuB2-9, NeuB2-13, NeuB2-19, NeuB2-29, NeuB2-37, NeuB2-40, NeuB2-55, NeuB2-60, NeuB2-80, NeuB2-93, NeuB2-110, or NeuB2-128 monoclonal antibody.

2. The antibody of claim 1, or antigen-binding fragment thereof, wherein the antibody comprises:
(a) a first V_{H} CDR at least 80% identical to the V_{H} CDR1 (SEQ ID NO: 1) of NeuB2-8, at least 80% identical to V_{H} CDR1 (SEQ ID NO: 4) of NeuB2-9, at least 80% identical to the V_{H} CDR1 (SEQ ID NO: 7) of NeuB2-13, at least 80% identical to the V_{H} CDR1 (SEQ ID NO: 10) of NeuB2-19, at least 80% identical to the V_{H} CDR1 (SEQ ID NO: 13) of NeuB2-29, at least 80% identical to the V_{H} CDR1 (SEQ ID NO: 16) of NeuB2-37, at least 80% identical to the V_{H} CDR1 (SEQ ID NO: 19) of NeuB2-40, at least 80% identical to the V_{H} CDR1 (SEQ ID NO: 22) of NeuB2-55, at least 80% identical to the V_{H} CDR1 (SEQ ID NO: 25) of NeuB2-60, at least 80% identical to the V_{H} CDR1 (SEQ ID NO: 28) of NeuB2-80, at least 80% identical to the V_{H} CDR1 (SEQ ID NO: 31) of NeuB2-93, at least 80% identical to the V_{H} CDR1 (SEQ ID NO: 34) of NeuB2-110, or at least 80% identical to the V_{H} CDR1 (SEQ ID NO: 37) of NeuB2-128;
(b) a second V_{H} CDR at least 80% identical to the V_{H} CDR2 (SEQ ID NO: 2) of NeuB2-8, at least 80% identical to the V_{H} CDR2 (SEQ ID NO: 5), at least 80% identical to the V_{H} CDR2 (SEQ ID NO: 8), at least 80% identical to the V_{H} CDR2 (SEQ ID NO: 11), at least 80% identical to the V_{H} CDR2 (SEQ ID NO: 14), at least 80% identical to the V_{H} CDR2 (SEQ ID NO: 17), at least 80% identical to the V_{H} CDR2 (SEQ ID NO: 20), at least 80% identical to the V_{H} CDR2 (SEQ ID NO: 23), at least 80% identical to the V_{H} CDR2 (SEQ ID NO: 26), at least 80% identical to the V_{H} CDR2 (SEQ ID NO: 29), at least 80% identical to the V_{H} CDR2 (SEQ ID NO: 32), at least 80% identical to the V_{H} CDR2 (SEQ ID NO: 35), or at least 80% identical to the V_{H} CDR2 (SEQ ID NO: 38);
(c) a third V_{H} CDR at least 80% identical to V_{H} CDR3 (SEQ ID NO: 3) of NeuB2-8, at least 80% identical to V_{H} CDR3 (SEQ ID NO: 6) of NeuB2-9, at least 80% identical to V_{H} CDR3 (SEQ ID NO: 9) of NeuB2-13, at least 80% identical to V_{H} CDR3 (SEQ ID NO: 12) of NeuB2-19, at least 80% identical to V_{H} CDR3 (SEQ ID NO: 15) of NeuB2-29, at least 80% identical to V_{H} CDR3 (SEQ ID NO: 18) of NeuB2-37, at least 80% identical to V_{H} CDR3 (SEQ ID NO: 21) of NeuB2-40, at least 80% identical to V_{H} CDR3 (SEQ ID NO: 24) of NeuB2-55, at least 80% identical to V_{H} CDR3 (SEQ ID NO: 27) of NeuB2-60, at least 80% identical to V_{H} CDR3 (SEQ ID NO: 30) of NeuB2-80, at least 80% identical to V_{H} CDR3 (SEQ ID NO: 33) of NeuB2-93, at least 80% identical to V_{H} CDR3 (SEQ ID NO: 36) of NeuB2-110, or at least 80% identical to V_{H} CDR3 (SEQ ID NO: 39) of NeuB2-128;
(d) a first V_{L} CDR at least 80% identical to V_{L} CDR1 (SEQ ID NO: 40) of NeuB2-8, at least 80% identical to V_{L} CDR1 (SEQ ID NO: 42) of NeuB2-9, at least 80% identical to V_{L} CDR1 (SEQ ID NO: 44) of NeuB2-13, at least 80% identical to V_{L} CDR1 (SEQ ID NO: 46) of NeuB2-19, at least 80% identical to V_{L} CDR1 (SEQ ID NO: 48) of NeuB2-29, at least 80% identical to V_{L} CDR1 (SEQ ID NO: 50) of NeuB2-37, at least 80% identical to V_{L} CDR1 (SEQ ID NO: 52) of NeuB2-40, at least 80% identical to V_{L} CDR1 (SEQ ID NO: 54) of NeuB2-55, at least 80% identical to V_{L} CDR1 (SEQ ID NO: 56) of NeuB2-60, at least 80% identical to V_{L} CDR1 (SEQ ID NO: 58) of NeuB2-80, at least 80% identical to V_{L} CDR1 (SEQ ID NO: 60) of NeuB2-93, at least 80% identical to V_{L} CDR1 (SEQ ID NO: 62) of NeuB2-110, or at least 80% identical to V_{L} CDR1 (SEQ ID NO: 64) of NeuB2-128;
(e) a second V_{L} CDR at least 80% identical to V_{L} CDR2 (tripeptide GVS) of NeuB2-8, at least 80% identical to V_{L} CDR2 (tripeptide SNN) of NeuB2-9, at least 80% identical to V_{L} CDR2 (tripeptide GAS) of NeuB2-13, at least 80% identical to V_{L} CDR2 (tripeptide DNN) of NeuB2-19, at least 80% identical to V_{L} CDR2 (tripeptide YDD) of NeuB2-29, at least 80% identical to V_{L} CDR2 (tripeptide SNN) of NeuB2-37, at least 80% identical to V_{L} CDR2 (tripeptide YDD) of NeuB2-40, at least 80% identical to V_{L} CDR2 (Tripeptide DVS) of NeuB2-55, at least 80% identical to V_{L} CDR2 (Tripeptide YDD) of NeuB2-60, at least 80% identical to V_{L} CDR2 (Tripeptide NNS) of NeuB2-80, at least 80% identical to V_{L} CDR2 (Tripeptide EVS) of NeuB2-93, at least 80% identical to V_{L} CDR2 (Tripeptide EVS) of NeuB2-110, or at least 80% identical to V_{L} CDR2 (Tripeptide DAS) of NeuB2-128; and
(f) a third V_{L} CDR at least 80% identical to V_{L} CDR3 (SEQ ID NO: 41) of NeuB2-8, at least 80% identical to V_{L} CDR3 (SEQ ID NO: 43) of NeuB2-9, at least 80% identical to V_{L} CDR3 (SEQ ID NO: 45) of NeuB2-13, at least 80% identical to V_{L} CDR3 (SEQ ID NO: 47) of NeuB2-19, at least 80% identical to V_{L} CDR3 (SEQ ID NO: 80) of NeuB2-29, at least 80% identical to V_{L} CDR3 (SEQ ID NO: 51) of NeuB2-37, at least 80% identical to V_{L} CDR3 (SEQ ID NO: 53) of NeuB2-40, at least 80% identical to V_{L} CDR3 (SEQ ID NO: 55) of NeuB2-55, at least 80% identical to V_{L} CDR3 (SEQ ID NO: 57) of NeuB2-60, at least 80% identical to V_{L} CDR3 (SEQ ID NO: 59) of NeuB2-80, at least 80% identical to V_{L} CDR3 (SEQ ID NO: 61) of NeuB2-93, at least 80% identical to V_{L} CDR3 (SEQ ID NO: 63) of NeuB2-110, or at least 80% identical to V_{L} CDR3 (SEQ ID NO: 65) of NeuB2-128.

3. The antibody of claim 2, wherein the antibody comprises:
(i) a V_{H} domain at least about 80% identical to the V_{H} domain (SEQ ID NO: 66) of NeuB2-8 or the humanized V_{H} domain of NeuB2-8 mAb; and a V_{L} domain at least about 80% identical to the V_{L} domain (SEQ ID NO: 79) of NeuB2-8 or the humanized V_{L} domain of NeuB2-8 mAb;
(ii) a V_{H} domain at least about 80% identical to the V_{H} domain (SEQ ID NO: 67) of NeuB2-9 or the humanized V_{H} domain of NeuB2-9 mAb; and a V_{L} domain at least about 80% identical to the V_{L} domain (SEQ ID NO: 80) of NeuB2-9 or the humanized V_{L} domain of NeuB2-9 mAb;
(iii) a V_{H} domain at least about 80% identical to the V_{H} domain (SEQ ID NO: 68) of NeuB2-13 or the humanized V_{H} domain of NeuB2-13 mAb; and a V_{L} domain at least about 80% identical to the V_{L} domain (SEQ ID NO: 81) of NeuB2-13 or the humanized V_{L} domain of NeuB2-13 mAb;
(iv) a V_{H} domain at least about 80% identical to the V_{H} domain (SEQ ID NO: 69) of NeuB2-19 or the humanized V_{H} domain of NeuB2-19 mAb; and a V_{L} domain at least about 80% identical to the V_{L} domain (SEQ ID NO: 82) of NeuB2-19 or the humanized V_{L} domain of NeuB2-19 mAb;
(v) a V_{H} domain at least about 80% identical to the V_{H} domain (SEQ ID NO: 70) of NeuB2-29 or the humanized V_{H} domain of NeuB2-29 mAb; and a V_{L} domain at least about 80% identical to the V_{L} domain (SEQ ID NO: 83) of NeuB2-29 or the humanized V_{L} domain of NeuB2-29 mAb;
(vi) a V_{H} domain at least about 80% identical to the V_{H} domain (SEQ ID NO: 71) of NeuB2-37 or the humanized V_{H} domain of NeuB2-37 mAb; and a V_{L} domain at least about 80% identical to the V_{L} domain (SEQ ID NO: 84) of NeuB2-37 or the humanized V_{L} domain of NeuB2-37 mAb;
(vii) a V_{H} domain at least about 80% identical to the V_{H} domain (SEQ ID NO: 72) of NeuB2-40 or the humanized V_{H} domain of NeuB2-40 mAb; and a V_{L} domain at least about 80% identical to the V_{L} domain (SEQ ID NO: 85) of NeuB2-40 or the humanized V_{L} domain of NeuB2-40 mAb;
(viii) a V_{H} domain at least about 80% identical to the V_{H} domain (SEQ ID NO: 73) of NeuB2-55 or the humanized V_{H} domain of NeuB2-55 mAb; and a V_{L} domain at least about 80% identical to the V_{L} domain (SEQ ID NO: 86) of NeuB2-55 or the humanized V_{L} domain of NeuB2-55 mAb;
(ix) a V_{H} domain at least about 80% identical to the V_{H} domain (SEQ ID NO: 74) of NeuB2-60 or the humanized V_{H} domain of NeuB2-60 mAb; and a V_{L} domain at least about 80% identical to the V_{L} domain (SEQ ID NO: 87) of NeuB2-60 or the humanized V_{L} domain of NeuB2-60 mAb;
(x) a V_{H} domain at least about 80% identical to the V_{H} domain (SEQ ID NO: 75) of NeuB2-80 or the humanized V_{H} domain of NeuB2-80 mAb; and a V_{L} domain at least about 80% identical to the V_{L} domain (SEQ ID NO: 88) of NeuB2-80 or the humanized V_{L} domain of NeuB2-80 mAb;
(xi) a V_{H} domain at least about 80% identical to the V_{H} domain (SEQ ID NO: 76) of NeuB2-93 or the humanized V_{H} domain of NeuB2-93 mAb; and a V_{L} domain at least about 80% identical to the V_{L} domain (SEQ ID NO: 89) of NeuB2-93 or the humanized V_{L} domain of NeuB2-93 mAb;
(xii) a V_{H} domain at least about 80% identical to the V_{H} domain (SEQ ID NO: 77) of NeuB2-110 or the humanized V_{H} domain of NeuB2-110 mAb; and a V_{L} domain at least about 80% identical to the V_{L} domain (SEQ ID NO: 90) of NeuB2-110 or the humanized V_{L} domain of NeuB2-110 mAb, or
(xiii) a V_{H} domain at least about 80% identical to the V_{H} domain (SEQ ID NO: 78) of NeuB2-128 or the humanized V_{H} domain of NeuB2-128 mAb; and a V_{L} domain at least about 80% identical to the V_{L} domain (SEQ ID NO: 91) of NeuB2-128 or the humanized V_{L} domain of NeuB2-128 mAb.

4. The antibody of any one of claims 1-3, wherein the antibody is recombinant.

5. The antibody of any one of claims 1-3, wherein the antibody is a human, humanized antibody or de-immunized antibody.

6. The antibody of any one of claims 1-3, wherein the antibody is conjugated to an imaging agent.

7. A chimeric antigen receptor comprising an antigen-binding domain at least 80% identical to an antigen-binding domain of the monoclonal antibody of any one of the preceding claims.

8. A composition comprising an antibody of any one of claims 1-6 in a pharmaceutically acceptable carrier.

9. An isolated polynucleotide molecule comprising a nucleic acid sequence encoding an antibody of any one of claims 1-6.

10. A host cell comprising one or more polynucleotide molecule(s) encoding an antibody of any one of claims 1-6.

11. The host cell of claim 10, wherein the host cell is a mammalian cell, a yeast cell, a bacterial cell, a ciliate cell or an insect cell.

12. (canceled)

13. A method of manufacturing an antibody comprising:
(a) expressing one or more polynucleotide molecule(s) encoding a V_{L} and V_{H} chain of an antibody of any one of claims 1-6 in a cell; and
(b) purifying the antibody from the cell.
